(19) United States
(12) Patent Application Publication
(10) Pub. No.: US 2008/0206283 A1
(43) Pub. Date: Aug. 28, 2008

(54) Vaccine Against Sars

(76) Inventors: Xiangjun Zhou, Guangdong Province (CN); John Y. Dong, Mount Pleasant, SC (US)

Correspondence Address: SCULLY SCOTT MURPHY & PRESSER, PC, 400 GARDEN CITY PLAZA, SUITE 300, GARDEN CITY, NY, 11530, US

(21) Appl. No.: 10/561,315

(22) PCT Filed: Jun. 17, 2004
(86) PCT No.: PCT/CN2004/000645

§ 371 Date: Apr. 4, 2008

(30) Foreign Application Priority Data
Jun. 17, 2003 (CN) 03142873.8

(51) Int. Cl. A61K 39/235; C07H 21/04; C07K 14/075; A61K 39/23; C12N 7/01

(52) U.S. Cl. 424/233.1; 536/23.1; 530/324; 530/350; 424/204.1; 435/235.1

(57) ABSTRACT

The present invention provides nucleotide sequences from SARS (Serve Acute Respiratory Syndrome) coronavirus genomes, as well as the applications of the partial fragments thereof in preparing DNA vaccine or expressing corresponding proteins. Furthermore, the present invention also provides the uses of said proteins in preventing and treating diseases, and preparing antibodies.

### FIELD OF THE INVENTION

The present invention relates to virology, especially to the nucleotide sequences of the SARS coronavirus, and the use of some of these sequences for DNA vaccine preparation and related proteins expression. It is also related to the use of those proteins in treatment and prevention of diseases.

### BACKGROUND OF THE INVENTION

The first case of Severe Acute Respiratory Syndrome (SARS) was found in November 2002, Guangdong Province, China. The number of the infected people and districts are increased over a six month period. According to the World Health Organization website, the number of reported cases is 8404, which includes patients from 32 countries and districts. Among them, 779 patients died.

SARS is a new highly contagious respiratory disease. It is different from Atypical Pneumonia (ATP) which is curable and less life-threatening. However, SARS could cause respiratory difficulties, which cause die. A new coronavirus was discovered through research of SARS, which is named as SARS coronavirus.

Coronavirus was first isolated from chickens in 1937. It was observed to have coronal spikes on its outer membrane under Electronic Microscope by a scientist named Rui Qin. Thus, it is named as “coronavirus”.

The Coronaviridea family was named by the Virus Naming Organization in 1975. It was classified as Coronavirus and torovirus according to its serological features and nucleotide sequences.

The classification of coronavirus:

The most common strain is Avian infectious bronchitis virus, IBV.

Other members are:
Human coronavirus
Murine virus hepatitis, MHV
Porcine hemagglutinating encepha lomyelitis virus
Porcine transmissible gastroenteritis virus, TGEV
Neonatal calf diarrhea coronavirus, BCV
Rat coronavirus, RCV
Turkey bluecomb virus
Feline infectious peritonitis virus

Possible members are:
Canine coronavirus
Sialodacryoadenitis virus of rat
Human enteric coronavirus

The physical and chemical characteristics of coronavirus include a diameter of 60-220 nm, and the coronal form. The coronavirus only comprises RNA as the genetic substance, the RNA and N protein composing its primary structure. It has 3 structural proteins, which are all glycoproteins. Its RNAs have very high recombination rate which contributes to the mutations, because the recombinations change the RNA sequences and amino acid sequences of proteins.

The epidemiology of the coronavirus:

As of this manuscript, 15 kinds of coronaviruses have been found. Some of those could cause diseases in human being, some in animals, including cows, pigs, rats, cats, dogs and birds. Two especially prevalent diseases are pestilence of chicken and dog. The pestilence of dog is an acute gastrointestinal infection and diarrhea is the clinical condition. The pathogen is coronavirus, which dwells in gastrointestine of the sick dog, comes out in fecal matter and contaminates the environment and feed. Thus, the canine coronavirus mainly infects by the digestive apparatus. The virus has strong resistance to external environment. It can survive 6˜9 days in fecal matter and several days in water. The pestilence of dog could not be easily controlled in a short period of time in an outbreak. The virus is sensitive to heat, ultra-violet radiation, lysol, 0.1% peracetic acid and 1% keliaonin.

Coronavirus can infect human, poultry and livestock. It could cause contagious bronchitis of poultry, hepatitis of rat, encephalomyelitis of pig and contagious peritonitis of cat. It could also cause respiratory infection and intestinal infection in humans.

It is very hard to separate or reproduce the coronavirus because human splanchnic cells, trachea cells, and nasal mucosa cells are required in organic culture.

This virus is very sensitive to temperature. It grows well at 33° C., and is inhibited at 35° C. Thus the diseases caused by this virus usually break out in winter and early spring.

There is currently no specific medicine of prevention or treatment for coronavirus.

Specific prevention, i.e. the application of vaccine, is possible but time consuming. And the reproduction of the virus could be a bottle neck for this solution.

Non-specific prevention, which is the method for prevention of spring respiratory tract diseases published by the World Health Organization, includes keeping warm, hand washing, airing, non-fatigue, preventing contact with patients, and avoiding public places. Treatment is specific for the disease. Coronavirus is very popular all over the world. Humans usually contain antibodies for coronavirus. Adults have higher amounts of antibodies than children. The coronavirus antibody rate of the group is different among different countries. The antibody rate reported in China is 30%˜60%. The coronavirus of respiratory tract infections are spread in the air. The infection rate peaks in fall, winter and early spring. It was reported that there are different infection cycles for different viruses, the breaking has 2˜3 years interval. The immunological reaction stimulated by coronavirus is weak and re-infection is common.

The clinical features of the coronavirus:

The coronavirus is the main pathogen for the adult cold. It could cause upper respiratory tract infection in children, yet it would seldom cause the lower respiratory tract infection. The incubation period of coronavirus is usually 2˜5 days, and approximately 3 days on average. The typical symptoms of coronavirus infection include cold symptoms such as nose running and uneasiness. The pathogenic ability and the clinical condition vary in different coronaviruses. The condition of OC43 is more serious than the one of 229E. There have been reports that coronavirus could cause fever, shivering, and vomiting lasting about 1 week. The clinical symptoms are not serious and no after effects are known.

Coronavirus could also cause infant acute gastrointestinal infections. The major symptoms include liquid bowel movements, fever and vomiting up to 10 times per day. Blood stained liquid fecal matter could occur in serious cases.

The clinical symptoms of coronavirus infections described in documentations include:

1) respiratory system infection, including SARS;

2) intestinal infection (occurs occasionally in babies);

3) nervous system symptoms (very rare);

Coronavirus comes out of the human body through respiratory tract secretions. It is spread out through saliva, breathing, and physical contact. Many of the coronaviruses cause non-serious and self-healing diseases. Nervous system symptoms could occur in rare cases.
The pathogen of SARS was proven to be mutation of the coronavirus by the World Health Organization on Apr. 16, 2003. It was named “SARS coronavirus”. It is closely related to the flu virus, however, it has unique qualities and was never found in humans in the past. As described before, coronavirus is spherical membrane virus having positive RNA strand with a diameter of 80˜220 nm and coronal spikes on its outer membrane. Further research indicated that two glycoproteins, including S and M protein, were found in the viral membrane. S protein could induce merging between the viral membrane and the host cell membrane, thereby leading to humoral and cellular immunoresponse. The viral RNA has a length of 26˜32 kb, which is the longest among all viral RNAs. The other N protein is nuclear capsid protein, which is related to RNA's reproduction and viral budding. RNA polymerase was first translated and produced when the host cell was infected by the coronavirus. The early events of infection were directed by the RNA polymerase. Then a series of transcription, replication, translation and reproduction of new virus start. A number of Open Reading Frames (ORF) were included in viral genes, and repetitive and separation sections were included in the ORFs. The sequence of all the coronavirus genes including SARS coronavirus was consistent, which is: 5′-RNA polymerase gene-S protein gene-E protein gene-M protein gene-N protein gene-3′. However, the SARS was different from other known coronaviruses. The known coronaviruses could infect humans, as well as many other animals within their respiratory tracts, digestive tracts, liver and nervous system. The coronavirus could be classified into 3 groups according to its immunology and sequence identity of nucleotide sequence as follows: Group 1 includes human respiratory tract coronavirus 229E, porcine transmissible gastroenteritis virus, feline enteric virus and canine coronavirus; Group 2 includes human respiratory coronavirus OC43, cow coronavirus, and porcine hemagglutinating encepha lomyelitis virus; Group 3 includes avian infectious bronchitis virus. After comparison of the gene sequences of the SARS coronavirus to those of the known 3 groups of coronavirus, and investigation into the systematic evolutionary tree of a few of the most important structural proteins, it was found that SARS coronavirus was not closely related to any of the other coronaviruses.

As the pathogen of an acute contagious disease, SARS coronavirus has a very high mutation rate. It is very important to investigate the genetic information, structure, and reproduction cycle of this pathogen for producing vaccine and medicine. Diagnostic medicines and methods are highly needed.

### SUMMARY OF INVENTION

The invention provided a method for sequencing the SARS coronavirus genes, and some applications of this sequence.

1. First, the SARS coronavirus genetic sequences were provided in the invention. The total RNA was obtained from infected tissues of the died Atypical Pneumonia patients. cDNA was then obtained by transcription. After the genome of virus gene was sequenced, it was found that the number of nucleotides in virus gene was 29760 which was shown in SEQ ID NO:1. It was indicated in the applicant's priority that 15 nucleotides were missing at the 5′ end, which means that 29745 nucleotides were reported. The genome sequences of the SARS coronavirus provided in the invention were recorded in GenBank, Accession No. AY390556 [gi: 41323719].

It was indicated in the initial analysis of the genome sequences of SARS virus that at least 11 ORFs were included, which express virus spike protein S, membrane protein M, envelope protein E, nuclear capsid protein N, and orflab, which could generate several proteins. Among these, S was a very important epitope protein. S and M were first inserted into endoplasmic reticulum while N was connected with the replicated RNA. Then the combination of protein-RNA was connected with protein M and entered the endoplasmic reticulum (see Tin-Yun Ho, Shih-Lu Wu, et al., Antigenicity and receptor-binding ability of recombinant SARS coronavirus spike protein. Biochemical and Biophysical Research Communications 313, 2004, 938-947).

2. Isolated polynucleotides were provided in the invention. The polynucleotides included the following: a) a polynucleotide sequence of SEQ ID NO:1; b) a naturally-occurring polynucleotide sequence having at least 90% sequence identity to the sequence of SEQ ID NO:1; and c) a polynucleotide sequence complementary to either a) or b).

3. The isolated polynucleotides provided in the invention were PCR amplified by using the primer described below, the template of gene sequence of SARS coronavirus.

1^{st} group of primers: upstream primer 5′ ACA GGA TCC AAG AAC ATG TTT ATT TTC TTA TT 3′, downstream primer 5′ AGA TCT GAA TTC TAT CCA ATA GGA ATG TCG CAC TC 3′;

2^{nd} group of primers: upstream primer 5′ ATT GGA TCC ACC ATG GGC TGT CTT ATA GGA GCT GAG C 3′, downstream primer 5′ ATG GAT CCG AAT TCT GGC TGT GCA GTA ATT GAT CT 3′;

3^{rd} group of primers: upstream primer 5′ CAA GGA TCC GTT ATG TAC TCA TTC GTT TCG 3′, downstream primer 5′ ACA AGA TCT GAA TTC TTT AAG CTC CTC AAC GGT AA 3′;

4^{th} group of primers: upstream primer 5′ ACA GGA TCC ATC ATG GCA GAC AAC GGT AC 3′, downstream primer 5′ AAC AGA TCT GAA TTC GCA ATC CTG AAA GTC CTC ATA 3′;

5^{th} group of primers: upstream primer 5′ ATT GGA TCC GTC ATG GAC AAT AAC CAG AAT GGA GGA CG 3′, downstream primer 5′ AAC AGA TCT GAA TTC ATT CTG CAC AAG AG 3′;

6^{th} group of primers: upstream primer 5′ ACA CCA TGG AAT TCG ACA TGG CTA TTT CAC CGA AG 3′, downstream primer 5′ CAG GTA CCG GAT CCA ATA TTG CAG CAG TAC GCA C 3′.

The template of the amplification was a molecule, such as cDNA, having the nucleotide sequence described in SEQ ID NO:1. The methods and conditions of the amplification are well known in the technical field, referenced in “Molecular Cloning Experimental Guide” (J Sambrook E. F. Fritsch T. Maniatis, Molecular Cloning, a Laboratory Manual, 2^{nd} ed, Cold Spring Harbor Laboratory Press, 1989).

4. The isolated polypeptide provided in the invention was translated by the genome sequence of SARS coronavirus mentioned in item 1, that is, the polypeptide was translated by the polynucleotide sequence in SEQ ID NO:1.

5. The isolated polypeptides provided in the invention was translated by the isolated polynucleotide described in item 3.

6. The isolated polynucleotide provided in the invention has at least 90% sequence identity to the naturally occurring nucleotide sequence described in item 3.

7. The isolated polypeptide provided in the invention has at least 90% sequence identity to the naturally occurring nucleotide sequence described in item 4.

8. An antibody which specifically binds to the mentioned isolated polypeptide fragment is provided in the invention, and it was a mono-clonal antibody in one embodiment.

9. A pharmaceutical composition which includes the polynucleotide, polypeptide, and the pharmaceutically acceptable carrier is provided in the invention.

10. A diagnostic kid having the polynucleotide of the present invention is provided in the invention.

11. A recombinant adenovirus which contains the polynucleotide is provided in the invention.

12. A vaccine which contains the adenovirus in item 11 is provided in the invention.

The above description is a concise summary of the invention. However, the invention was not limited to that. The rest of the invention, simple modifications and improvements based on the invention, are all included in the invention.

It was found in one of the embodiments that the immunological reaction for the SARS coronavirus could be induced in vivo by 6 polypeptides or protein fragments. Thus those fragments could be used as vaccines. They were polynucleotides by PCR amplified using 6 groups of primers of item 3, templates of the genome sequences of SARS coronavirus. They were recorded respectively as SEQ ID No:2, SEQ ID No:3, SEQ ID No:4, SEQ ID No: 5, SEQ ID No:6 and SEQ ID No:7.

The nucleotide sequence of SEQ ID NO:1 for the vaccine, could also include sequences with more than 90% sequence identity to the nucleotide sequence of SEQ ID NO:1 for the vaccine. The preferred sequences are the nucleotide fragment of SEQ ID No:2, SEQ ID No:3, SEQ ID No:4, SEQ ID No: 5, SEQ ID No:6, SEQ ID No:7, or the sequence with more than 90% sequence identity to those sequence.

A protein vaccine was provided in another embodiment of the invention. This vaccine contained the polypeptide and protein fragments translated by the nucleotide sequence of SEQ ID NO:1.

The inventor noted that the translated product of SEQ ID NO:1 could cause immunological reactions. Any translation product of SEQ ID NO:1 was included in the invention. The translation products of SEQ ID NO:1, with different start site are also included in the invention. The whole amino acid sequence translated from SEQ ID NO:1 was recorded as SEQ ID NO:8.

The isolated polypeptide in the invention included the following amino acid sequences:
a) SEQ ID NO: 8;
b) a naturally-occurring amino acid sequence having at least 90% sequence identity to the sequence of SEQ ID NO:8;
c) a biologically-active fragment of the amino acid sequence of SEQ ID NO:8; and
d) an immunogenic fragment of the amino acid sequence of SEQ ID NO:8.

A protein vaccine is provided in another embodiment of the invention. The sequences included protein fragments translated by SEQ ID No:2, SEQ ID No:3, SEQ ID No:4, SEQ ID No: 5, SEQ ID No:6 and SEQ ID No:7. Those protein fragments were recorded as SEQ ID No:9, SEQ ID No:10, SEQ ID No:11, SEQ ID No: 12, SEQ ID No:13 and SEQ ID No:14.

The DNA or RNA fragments designed based on SEQ ID NO:1 in another embodiment, could be used as diagnostic probes or ingredient of the gene chips. Furthermore, these fragments could be used as treatment molecules, such as reverse RNA molecules, which could complement or was similar to part of the SARS coronavirus sequence or the gene sequence described in the invention. The gene sequences included fragments SEQ ID No:2, SEQ ID No:3, SEQ ID No:4, SEQ ID No: 5, SEQ ID No:6 and SEQ ID No:7. The nucleotide sequence or their fragments based on the genome sequence of the invention, which could combine with the virus gene to prohibit virus replication, transcription, and translation are included in the invention. The invention also included the use of vectors containing the nucleotide sequences or the nucleotide sequences themselves.

The nucleotides sequence was inserted in a vector in one of the embodiments. The vector could be of any type and be transfected into host cells. The host cells were either eukaryotic cells or prokaryotic cells. Thus the SARS virus proteins were expressed in host cells. The nucleotide probe including at least 15 nucleotides, could specifically hybridize with the nucleotide sequence of SEQ ID No:1.

The nucleotide probe could be flagged using testable markers which could be used in the diagnosis of SARS.

The gene sequencing of the invention could be used in PCR and immunology testing, thus assisting in diagnosis of SARS infections in human and potential animal hosts. It could help in developments of anti-viral medicines, including neutralizing antibodies, as well as in testing epitopes in development of vaccines. This genetic information could also assist in preparing gene chips for testing and diagnosis.

The specific 29 nucleotides in SARS coronavirus gene were provided in the invention. The specific sequence located in SEQ ID No:1 from 27891 to 27919, was named as SEQ ID No:15. Its sequence is as follows:

CCTACTGGTTACCAACCTGAATGGAATAT

The sequence described above could be used in preparation of a diagnosis kit.

47. The technical solution provided in the invention was summarized as follows:
1. An isolated polynucleotide selected from the group consisting of:
   a. a polynucleotide sequence of SEQ ID NO:1;
   b. a naturally-occurring polynucleotide sequence having at least 90% sequence identity to the sequence of SEQ ID NO:1; and
   c. a polynucleotide sequence complementary to either a) or b).
2. An isolated polynucleotide sequence encoding a polypeptide comprising an amino acid sequence selected from the group consisting of:
   a. SEQ ID NO: 8;
   b. a naturally-occurring amino acid sequence having at least 90% sequence identity to the sequence of SEQ ID NO:8;
   c. a biologically-active fragment of the amino acid sequence of SEQ ID NO:8; and
d. an immunogenic fragment of the amino acid sequence of SEQ ID NO:8.
3. An isolated polynucleotide selected from the group consisting of:
   a. a polynucleotide sequence selected from the group consisting of SEQ ID NOs: 2-7;
   b. a naturally-occurring polynucleotide sequence having at least 90% sequence identity to a sequence selected from the group consisting of SEQ ID NOs: 2-7; and
   c. a polynucleotide sequence complementary to either a) or b).
4. An isolated polypeptide sequence comprising an amino acid sequence selected from the group consisting of:
   a) an amino acid sequence of SEQ ID NO. 8;
   b) a naturally-occurring amino acid sequence having at least 90% sequence identity to the amino acid sequence of SEQ ID NO. 8;
   c) a biologically active fragment of the amino acid sequence of SEQ ID NO. 8; and
   d) an immunogenic fragment of the amino acid sequence of SEQ ID NO. 8.
5. An isolated polypeptide fragment capable of generating an immune response against the SARS virus selected from the group consisting of
   a. a polypeptide sequence selected from the group consisting of SEQ ID NOs: 9-14;
   b. a naturally-occurring polypeptide sequence having at least 90% sequence identity to a sequence selected from the group consisting of SEQ ID NOs: 9-14.
6. An isolated antibody which specifically binds to a polypeptide of claim 4.
7. An isolated antibody which specifically binds to a polypeptide of claim 5.
8. The isolated antibody of claim 6, wherein said antibody is a monoclonal antibody.
9. The isolated antibody of claim 7, wherein said antibody is a monoclonal antibody.
10. A pharmaceutical composition comprising an effective amount of the polypeptide of claim 4 and a pharmaceutically acceptable carrier.
11. A pharmaceutical composition comprising an effective amount of the polypeptide of claim 5 and a pharmaceutically acceptable carrier.
12. A pharmaceutical composition comprising an effective amount of the polynucleotide of claim 1 and a pharmaceutically acceptable carrier.
13. A pharmaceutical composition comprising an effective amount of the polynucleotide of claim 2 and a pharmaceutically acceptable carrier.
14. A pharmaceutical composition comprising an effective amount of the polynucleotide of claim 3 and a pharmaceutically acceptable carrier.
15. A pharmaceutical composition comprising the antibody of claim 6 in conjunction with a pharmaceutically acceptable carrier.
16. A pharmaceutical composition comprising the antibody of claim 7 in conjunction with a pharmaceutically acceptable carrier.
17. A pharmaceutical composition comprising the antibody of claim 8 in conjunction with a pharmaceutically acceptable carrier.
18. A pharmaceutical composition comprising the antibody of claim 9 in conjunction with a pharmaceutically acceptable carrier.
19. A diagnostic kit for detecting the presence of SARS virus in a sample comprising the polynucleotide of claim 1 and a pharmaceutically acceptable carrier.
20. A diagnostic kit for detecting the presence of SARS virus in a sample comprising the polynucleotide of claim 2 and a pharmaceutically acceptable carrier.
21. A diagnostic kit for detecting the presence of SARS virus in a sample comprising the polynucleotide of claim 3 and a pharmaceutically acceptable carrier.
22. A probe for use in detecting the presence of SARS virus in a sample comprising at least 20 contiguous polynucleotides comprising a sequence complementary to the SARS viral polynucleotide in the sample, and said probe specifically hybridizes to the SARS viral polynucleotide under conditions whereby a hybridization complex is formed between said probe and said SARS viral polynucleotide.
23. A probe for use in detecting the presence of a specific SARS virus in a sample comprising the polynucleotide sequence of SEQ ID NO: 15.
24. A method of detecting a SARS viral polynucleotide in a sample, said SARS viral polynucleotide having the sequence of the polynucleotide of claim 1, comprising:
   a. hybridizing the sample with a probe comprising at least 20 contiguous nucleotides comprising a sequence complementary to the SARS viral polynucleotide in the sample, and said probe specifically hybridizes to the SARS viral polynucleotide under conditions whereby a hybridization complex is formed between said probe and said SARS viral polynucleotide; and
   b. detecting the presence or absence of said hybridization complex, and optionally, if present, the amount thereof.
25. A method of detecting a SARS viral polynucleotide in a sample, said SARS viral polynucleotide having the sequence of the polynucleotide of claim 2, comprising:
   a. hybridizing the sample with a probe comprising at least 20 contiguous nucleotides comprising a sequence complementary to the SARS viral polynucleotide in the sample, and said probe specifically hybridizes to the SARS viral polynucleotide under conditions whereby a hybridization complex is formed between said probe and said SARS viral polynucleotide; and
   b. detecting the presence or absence of said hybridization complex, and optionally, if present, the amount thereof.
26. A method of detecting a SARS viral polynucleotide in a sample, said SARS viral polynucleotide having the sequence of the polynucleotide of claim 3, comprising:
   a. hybridizing the sample with a probe comprising at least 20 contiguous nucleotides comprising a sequence complementary to the SARS viral polynucleotide in the sample, and said probe specifically hybridizes to the SARS viral polynucleotide under conditions whereby a hybridization complex is formed between said probe and said SARS viral polynucleotide; and
   b. detecting the presence or absence of said hybridization complex, and optionally, if present, the amount thereof.
27. The method of claim 24 above, wherein the probe comprises at least 30 contiguous nucleotides.
28. The method of claim 25 above, wherein the probe comprises at least 30 contiguous nucleotides.
29. The method of claim 26 above, wherein the probe comprises at least 30 contiguous nucleotides.
30. The method of claim 24 above, wherein the probe comprising at least 50 contiguous nucleotides.
31. The method of claim 25 above, wherein the probe comprising at least 50 contiguous nucleotides.
32. The method of claim 26 above, wherein the probe comprising at least 50 contiguous nucleotides.
33. A method for detecting a polynucleotide which encodes a SARS virus protein in a biological sample comprising the steps of:
   a. hybridizing the polynucleotide of claim 1 to a nucleic acid material of a biological sample, thereby forming a hybridization complex; and
   b. detecting said hybridization complex, wherein the presence of said hybridization complex correlates with the presence of a polynucleotide encoding the SARS viral protein in said biological sample.
34. A method for detecting a polynucleotide which encodes a SARS virus protein in a biological sample comprising the steps of:
   a. hybridizing the polynucleotide of claim 2 to a nucleic acid material of a biological sample, thereby forming a hybridization complex; and
   b. detecting said hybridization complex, wherein the presence of said hybridization complex correlates with the presence of a polynucleotide encoding the SARS viral protein in said biological sample.
35. A method for detecting a polynucleotide which encodes a SARS virus protein in a biological sample comprising the steps of:
   a. hybridizing the polynucleotide of claim 3 to a nucleic acid material of a biological sample, thereby forming a hybridization complex; and
   b. detecting said hybridization complex, wherein the presence of said hybridization complex correlates with the presence of a polynucleotide encoding the SARS viral protein in said biological sample.

36. A vaccine effective against a human SARS virus infection comprising a peptide having a sequence selected from the group consisting of SEQ ID NOs: 1-7 and a pharmaceutically acceptable carrier.
37. A vaccine effective against a human SARS virus infection comprising a peptide having a sequence selected from the group consisting of SEQ ID NOs: 8-14 and a pharmaceutically acceptable carrier.
38. A recombinant adenovirus expressing SARS viral proteins, comprising:
   a. an adenovirus, wherein portions of its sequence responsible for replication having been deleted, thus rending the adenovirus incapable of replicating itself; and
   b. at least one polypeptide fragment selected from the group consisting of the spike protein, the small membrane protein, the small envelope protein, and the nuclear capsid protein.
39. A recombinant adenovirus expressing SARS viral proteins, comprising:
   a. an adenovirus, wherein portions of its sequence responsible for replication having been deleted, thus rending the adenovirus incapable of replicating itself; and
   b. two polypeptide fragments selected from the group consisting of the spike protein, the small membrane protein, the small envelope protein, and the nuclear capsid protein.
40. A recombinant adenovirus expressing SARS viral proteins, comprising:
   a. an adenovirus, wherein portions of its sequence responsible for replication having been deleted, thus rending the adenovirus incapable of replicating itself; and
   b. three polypeptide fragments selected from the group consisting of the spike protein, the small membrane protein, the small envelope protein, and the nuclear capsid protein.
41. A recombinant adenovirus expressing SARS viral proteins, comprising:
   a. an adenovirus, wherein portions of its sequence responsible for replication having been deleted, thus rending the adenovirus incapable of replicating itself; and
   b. a plurality of polypeptide fragments selected from the group consisting of the spike protein, the small membrane protein, the small envelop protein, and the nuclear capsid protein.
42. A recombinant adenovirus expressing SARS viral proteins, comprising:
   a. an adenovirus, wherein portions of its sequence responsible for replication having been deleted, thus rending the adenovirus incapable of replicating itself;
   b. the spike protein of the SARS virus; and
   c. the small envelop protein.
43. A recombinant adenovirus expressing SARS viral proteins, comprising:
   a. an adenovirus, wherein portions of its sequence responsible for replication having been deleted, thus rending the adenovirus incapable of replicating itself;
   b. the spike protein of the SARS virus; and
   c. the small membrane protein.
44. A recombinant adenovirus expressing SARS viral proteins, comprising:
   a. an adenovirus, wherein portions of its sequence responsible for replication having been deleted, thus rending the adenovirus incapable of replicating itself;
   b. the spike protein of the SARS virus;
   c. the small membrane protein; and
   d. the small envelop protein.
45. A recombinant adenovirus expressing SARS viral proteins, comprising:
   a. an adenovirus, wherein portions of its sequence responsible for replication having been deleted, thus rending the adenovirus incapable of replicating itself;
   b. the small envelope protein;
   c. the small membrane protein; and
   d. the nuclear capsid protein.
46. A SARS vaccine comprising of the recombinant adenovirus of claim 38, and a pharmaceutically acceptable carrier.
47. A SARS vaccine comprising of the recombinant adenovirus of claim 39, and a pharmaceutically acceptable carrier.
48. A SARS vaccine comprising of the recombinant adenovirus of claim 40, and a pharmaceutically acceptable carrier.
49. A SARS vaccine comprising of the recombinant adenovirus of claim 41, and a pharmaceutically acceptable carrier.
50. A SARS vaccine comprising of the recombinant adenovirus of claim 42, and a pharmaceutically acceptable carrier.
51. A SARS vaccine comprising of the recombinant adenovirus of claim 43, and a pharmaceutically acceptable carrier.
52. A SARS vaccine comprising of the recombinant adenovirus of claim 44, and a pharmaceutically acceptable carrier.
53. A SARS vaccine comprising of the recombinant adenovirus of claim 45, and a pharmaceutically acceptable carrier.
54. A method of modulating the immune response to human SARS virus infection, comprising administering an effective amount of the vaccine according to claim 46.
55. A method of modulating the immune response to human SARS virus infection, comprising administering an effective amount of the vaccine according to claim 47.
56. A method of modulating the immune response to human SARS virus infection, comprising administering an effective amount of the vaccine according to claim 48.
57. A method of modulating the immune response to human SARS virus infection, comprising administering an effective amount of the vaccine according to claim 49.
58. A method of modulating the immune response to human SARS virus infection, comprising administering an effective amount of the vaccine according to claim 50.
59. A method of modulating the immune response to human SARS virus infection, comprising administering an effective amount of the vaccine according to claim 51.
60. A method of modulating the immune response to human SARS virus infection, comprising administering an effective amount of the vaccine according to claim 52.
61. A method of modulating the immune response to human SARS virus infection, comprising administering an effective amount of the vaccine according to claim 53.
62. A method of immunizing a subject against a SARS virus infection comprising administering to said subject the vaccine of claim 46.
63. A method of immunizing a subject against a SARS virus infection comprising administering to said subject the vaccine of claim 47.
64. A method of immunizing a subject against a SARS virus infection comprising administering to said subject the vaccine of claim 48.
65. A method of immunizing a subject against a SARS virus infection comprising administering to said subject the vaccine of claim 49.
66. A method of immunizing a subject against a SARS virus infection comprising administering to said subject the vaccine of claim 50.
67. A method of immunizing a subject against a SARS virus infection comprising administering to said subject the vaccine of claim 51.
68. A method of immunizing a subject against a SARS virus infection comprising administering to said subject the vaccine of claim 52.
69. A method of immunizing a subject against a SARS virus infection comprising administering to said subject the vaccine of claim 53.
70. The method of claim 62, wherein said subject is a human.
71. The method of claim 63, wherein said subject is a human.
72. The method of claim 64, wherein said subject is a human.
73. The method of claim 65, wherein said subject is a human.
74. The method of claim 66, wherein said subject is a human.
75. The method of claim 67, wherein said subject is a human.
76. The method of claim 68, wherein said subject is a human.
77. The method of claim 69, wherein said subject is a human.
78. A method of treating a SARS virus infection in a subject comprising administering to said subject the vaccine of claim 46.
79. A method of treating a SARS virus infection in a subject comprising administering to said subject the vaccine of claim 47.
80. A method of treating a SARS virus infection in a subject comprising administering to said subject the vaccine of claim 48.
81. A method of treating a SARS virus infection in a subject comprising administering to said subject the vaccine of claim 49.
82. A method of treating a SARS virus infection in a subject comprising administering to said subject the vaccine of claim 50.
83. A method of treating a SARS virus infection in a subject comprising administering to said subject the vaccine of claim 51.
84. A method of treating a SARS virus infection in a subject comprising administering to said subject the vaccine of claim 52.
85. A method of treating a SARS virus infection in a subject comprising administering to said subject the vaccine of claim 53.
86. The method of claim 78, wherein said subject is a human.
87. The method of claim 79, wherein said subject is a human.
88. The method of claim 80, wherein said subject is a human.
89. The method of claim 81, wherein said subject is a human.
90. The method of claim 82, wherein said subject is a human.
91. The method of claim 83, wherein said subject is a human.
92. The method of claim 84, wherein said subject is a human.
93. The method of claim 85, wherein said subject is a human.

### DETAILED DESCRIPTION OF THE INVENTION

The inventor had participated in the pathological research of the SARS patient on Jan. 31, 2003, and in the anatomy research on the same patient passed away on Feb. 10, 2003. The methods included: anatomy on the body with atypical pneumonia, infected tissues were sliced and observed under electron microscope, cDNA were obtained from total RNA obtained from infected tissues, SARS coronavirus full gene were sequenced. As a result, large area of infection, pulmonary edema, bleeding, focal haemorrhagic infarction were observed in lung tissues. The virus pellets were observed under electron microscope in II type alveolus epithelium cells. The full SARS coronavirus gene (named as GZ02102003), was named as SEQ ID NO:1 in the invention.

The detail research of the inventor was as follows:
1.1 Patient: passed away, female, aged 62, Guangzhou citizen, infected on Jan. 31, 2003, symptoms including fever, nose running, and sore throat and coughing. The symptom went severe on February 4 showing symptom of difficulties in breathing. She was diagnosed to have atypical pneumonia and transferred to Guangzhou 8^{th} People's Hospital. The symptoms were not relieved and the patient passed away at 00:15 on February 10. The inventors arrived in the next day and the body was dissected (at 15:00) on Feb. 11, 2003 in the South Hospital of the First Military Medical University.
1.2 Observation using Electron Microscope: the lung tissue was stabilized using 1% osmic acid for 30 minutes, washed with PBS, dehydrated using gradient acetone, imbedded using epoxy resin, thin slided, double stained using Uranium and Lead, and observed under electron microscope.
1.3 Full sequencing and analysis
   1.3.1 Extraction of total RNA: TRIZOL Reagent kit of Invitrogen corp. was used. The process was referred to the description of the kit.
   1.3.2 Transcription and sequencing of the full cDNA: cDNA was obtained by ThermoScript (Invitrogen, USA) and random primers. The PCR primers were designed according to the published SARS coronavirus full sequence. 1 kb length of the product was amplified using each pair of PCR primers. There was repetitive section of about 200 bases for each pair of primers next to each other. The whole PCR reaction had 39 cycles and volume of 25 μl. The reaction condition included annealing in the first 14 cycles and 0.5° C. decrease for each cycle. ABI Big Dye Terminator preparation was used on ABI377 equipment for PCR product sequencing. The assembling software for the product sequencing was the Phred, Phrap and Consed of University of Washington.
Results:
2.1 Anatomy: generalized infection in lung tissues (especially in bottom of the superior lobe of the left lung, inferior lobe of left lung, the right lung) included pulmonary edema, bleeding, and lung focal haemorrhagic infarction. Inside the infected alveolus, there were large amount of dropped and hyperplastic alveolus cells, dropsy liquid, a number of monocuclear phagocyte cells and lymphatic cells invasion in alveolar septum and inside alveolar, neutrophil invasion in pleura and part of the alveolars. Alveolars in both lungs became transparent and had necrosis in focal alveolar septum. Virus inclusion bodies were obtained from part of the alveolar cells. Lung bronchit cells were observed falling off. Lymphatic cells and mononuclear cells invasions were observed in part of the alveolar cells. It was also observed pulmonary fibrosis hyperemia, bleeding, capillarectasis, mononuclear cells, lymphatic cells and neutrophil in alveolar pulmonary, swelling of endothelial cells in pulmonary small arteries and veins, hyperplasia, endothelial dropsy, mononuclear cells and lymphatic cells invasion in middle and outside tunic of the blood vessels, transparent thrombotics in some of the blood vessels, high degree of expansion and hyperemia of blood vessels in pulmonary lymph, ambiguous outline of cortex and medulla, many mononuclear like cells in second cortex, reducing of lymphatic tissues in medulla, hyperemia of 200 ml in thorax, and thrombotics in pulmonary major artery.
2.2 Observation using electron microscope: coronal virus pellets were observed in II type alveolus epithelium cells (see FIG. 1).
2.3 Sequencing and analysis: The full SARS coronavirus sequence had 29760 bases, named as GZ02102003, which indicated that the sequence was obtained from the pulmonary tissue of a patient who passed away on Feb. 10, 2003. It was found that an extra nucleotide fragment of 29 nucleotides (CCTACTGGTTACCAACCTGAATGGAATAT) exists in this sequence, except for a few SNP, after comparing this sequence with other SARS coronavirus full sequences recorded in Genebank. There were 17 SARS coronavirus full sequences recorded in GeneBank until Jun. 6, 2003. However, there were many obvious mistakes in sequence ZJ01, which was then not included in the sequence comparisons. The results of the comparison were shown in table 1. full sequence comparison of 17 SARS coronavirus. The existence of this 29 nucleotide sequences, completely changed the coding frame for protein ORF10 and ORF11. This sequence also existed in civet SARS virus. However, this sequence of 29 nucleotides was missing from the SARS coronavirus from the patients infected after March 2003. This fact indicated that the SARS coronavirus isolated from the patient infected in January highly related to the civet SARS coronavirus. Thus, the inventor believed that the SARS infecting human was originated from civet.

TABLE 1
Full sequenc comparison of 17 SARS coronavirus

| | | | | | | | | | | | | | | | | | |
|---|---|---|---|---|---|---|---|---|---|---|---|---|---|---|---|---|---|
| 473 | 494 | 502 | 509 | 652 | 937 | 1180 | 1206 | 1384 | 1476 | 2601 | 3165 | 3274 | 3326 | 3626 | 4220 | 4250 | 4876 |

| | | | | | | | | | | | | | | | | | | |
|---|---|---|---|---|---|---|---|---|---|---|---|---|---|---|---|---|---|---|
| TOR2 | T | T | A | G | G | A | G | T | C | A | T | A | A | T | T | A | T | T |
| CUHK-SU10 | T | T | A | G | G | A | G | T | C | A | T | A | A | T | T | A | T | T |
| SIN2748 | T | T | A | G | G | A | G | T | C | A | T | A | A | T | T | A | T | T |
| SIN2500 | T | T | A | G | G | A | G | T | C | A | T | A | A | T | T | A | T | T |
| HKU-39849 | T | T | A | G | G | A | G | T | C | A | C | A | A | T | T | A | T | T |
| Urbani | T | T | A | G | G | A | G | T | C | A | T | A | A | T | T | A | T | T |
| TW1 | T | T | A | G | G | A | G | T | C | A | T | G | A | T | T | A | T | T |
| SIN2677 | T | T | A | G | G | A | G | T | C | G | T | A | A | T | T | A | T | T |
| SIN2774 | T | T | A | G | G | A | G | T | C | A | T | A | A | T | T | A | T | T |
| ZJ01 | G | A | T | G | G | A | G | T | M | A | T | A | A | T | T | A | T | T |
| SIN2679 | T | T | A | G | G | A | G | T | C | A | T | A | A | T | T | A | T | T |
| BJ01 | T | T | A | G | G | A | G | T | C | A | T | A | A | T | T | A | T | T |
| BJ03 | T | T | A | G | G | A | G | T | C | A | T | A | C | T | T | A | T | T |
| BJ02 | T | T | A | G | G | A | G | T | C | A | T | A | A | T | T | A | T | T |
| BJ04 | T | T | A | G | G | A | T | T | C | A | T | A | A | T | T | A | T | T |
| CUHK-W1 | T | T | A | G | G | A | G | T | C | A | T | A | A | T | T | A | T | T |
| GZ01 | T | T | A | T | A | C | G | T | C | A | T | A | A | T | C | G | C | A |
| GZ02102003 | T | T | A | T | G | A | G | C | C | A | T | A | A | C | C | G | T | T |

| | | | | | | | | | | | | | | |
|---|---|---|---|---|---|---|---|---|---|---|---|---|---|---|
| 4952 | 5251 | 5247 | 5547 | 5591 | 5594 | 5681 | 6148 | 6612 | 6929 | 7643 | 7665 | 7702 | 7731 | 7746 |

| | | | | | | | | | | | | | | | |
|---|---|---|---|---|---|---|---|---|---|---|---|---|---|---|---|
| TOR2 | T | C | A | A | A | A | G | T | G | G | C | T | C | C | G |
| CUHK-SU10 | T | C | A | A | A | A | G | T | G | G | C | T | C | C | G |
| SIN2748 | T | C | A | A | A | A | G | T | G | G | C | T | C | C | G |
| SIN2500 | T | C | A | A | A | A | G | T | G | G | C | T | C | C | G |
| HKU-39849 | T | C | A | A | A | A | G | T | G | G | C | T | C | C | G |
| Urbani | T | C | A | A | A | A | G | T | G | G | C | T | C | C | G |
| TW1 | T | C | A | A | A | A | G | T | G | G | C | T | C | C | G |
| SIN2677 | T | C | A | A | A | A | G | T | G | G | C | T | C | C | G |
| SIN2774 | T | C | A | A | A | A | G | T | G | G | C | T | C | C | G |
| ZJ01 | T | C | A | A | A | A | G | T | G | G | A | A | A | A | G |
| SIN2679 | T | C | A | A | A | A | G | T | G | G | C | T | C | C | G |
| BJ01 | T | C | A | A | A | A | G | T | G | G | C | T | C | C | G |
| BJ03 | T | C | C | C | C | C | G | T | G | G | C | T | C | C | G |
| BJ02 | A | C | A | A | A | A | T | T | G | G | C | T | C | C | G |
| BJ04 | T | C | A | A | A | A | G | A | G | G | C | T | C | C | G |
| CUHK-W1 | T | C | A | A | A | A | G | T | G | G | C | T | C | C | T |
| GZ01 | T | C | A | A | A | A | G | T | T | A | C | T | C | C | G |
| GZ02102003 | T | A | A | A | A | A | G | T | T | A | C | T | C | C | G |

| | | | | | | | | | | | | | | |
|---|---|---|---|---|---|---|---|---|---|---|---|---|---|---|
| 7919 | 7930 | 7954 | 8387 | 8417 | 8502 | 8559 | 8562 | 8573 | 8816 | 8947 | 9096 | 9177 | 9332 | 9333 |

| | | | | | | | | | | | | | | | |
|---|---|---|---|---|---|---|---|---|---|---|---|---|---|---|---|
| TOR2 | C | G | T | G | G | T | T | | G | C | T | C | T | T | C |
| CUHK-SU10 | C | G | T | G | G | T | T | | G | C | T | C | T | T | C |
| SIN2748 | C | G | T | G | G | T | T | | G | C | T | C | T | T | C |
| SIN2500 | C | G | T | G | G | T | T | | G | C | T | C | T | T | C |
| HKU-39849 | C | A | T | C | C | T | T | | G | C | T | C | T | T | C |
| Urbani | T | G | T | G | G | T | T | | G | C | T | C | T | T | C |
| TW1 | C | G | T | G | G | T | T | | G | C | T | C | T | T | C |
| SIN2677 | C | G | T | G | G | T | T | | G | C | T | C | T | T | C |
| SIN2774 | C | G | T | G | G | T | T | | G | C | T | C | T | T | C |
| ZJ01 | C | G | T | G | G | T | T | A | G | C | T | C | T | C | T |
| SIN2679 | C | G | T | G | G | T | T | | G | C | T | C | T | T | C |
| BJ01 | C | G | T | G | G | T | T | | T | C | T | C | T | T | C |
| BJ03 | C | G | T | G | G | T | T | | G | C | T | C | T | T | C |
| BJ02 | C | G | T | G | G | T | T | | T | C | T | C | T | T | C |
| BJ04 | C | G | T | G | G | T | T | | G | C | T | C | T | T | C |
| CUHK-W1 | C | G | T | G | G | T | T | | G | C | T | C | T | T | C |
| GZ01 | C | G | C | G | G | T | C | | G | T | A | T | C | T | C |
| GZ02102003 | C | G | T | G | G | G | C | | G | T | A | T | C | T | C |

| | | | | | | | | | | | |
|---|---|---|---|---|---|---|---|---|---|---|---|
| 9405 | 9480 | 9855 | 10030 | 10324 | 10551 | 10588 | 10729 | 11492 | 11718 | 11972 | 11975 |

| | | | | | | | | | | | | |
|---|---|---|---|---|---|---|---|---|---|---|---|---|
| TOR2 | T | T | C | G | A | A | A | C | T | A | G | A |
| CUHK-SU10 | T | T | C | G | A | A | A | C | T | A | G | A |
| SIN2748 | T | T | C | G | A | A | A | C | T | A | G | A |
| SIN2500 | T | T | C | G | A | A | A | C | T | A | G | A |
| HKU-39849 | T | T | C | G | A | A | A | C | T | A | G | A |
| Urbani | T | T | C | G | A | A | A | C | T | A | G | A |
| TW1 | T | T | C | G | A | A | A | C | T | A | G | A |
| SIN2677 | T | T | C | G | A | A | A | C | T | A | G | A |
| SIN2774 | T | T | C | G | A | A | A | C | T | A | G | A |
| ZJ01 | T | T | C | G | A | A | A | C | A | A | G | A |
| SIN2679 | T | T | C | G | A | A | A | C | T | A | G | A |
| BJ01 | C | T | T | G | A | A | C | C | T | A | G | A |
| BJ03 | C | T | T | G | A | A | A | C | T | A | A | A |
| BJ02 | C | T | T | G | A | G | A | C | T | A | G | T |
| BJ04 | T | T | T | G | A | A | A | A | T | C | G | A |
| CUHK-W1 | C | C | C | G | A | A | A | C | T | A | G | A |
| GZ01 | C | C | C | A | G | A | C | C | T | A | G | A |
| GZ02102003 | C | C | C | A | A | A | A | C | T | A | G | A |

| | | | | | | | | | | |
|---|---|---|---|---|---|---|---|---|---|---|
| 12151 | 12518 | 12519 | 12983 | 12989 | 12996 | 13027 | 13029 | 13388 | 13464 | 13476 |

| | | | | | | | | | | | |
|---|---|---|---|---|---|---|---|---|---|---|---|
| TOR2 | T | A | T | A | | C | C | A | | | |
| CUHK-SU10 | T | A | T | A | | C | C | A | | | |
| SIN2748 | T | A | T | A | | C | C | A | | | |
| SIN2500 | T | A | T | A | | C | C | A | | | |
| HKU-39849 | T | A | T | A | | C | C | A | | | |
| Urbani | T | A | T | A | | C | C | A | | | |
| TW1 | T | A | T | A | | C | C | A | | | |
| SIN2677 | T | A | T | A | | C | C | A | | | |
| SIN2774 | T | A | T | A | | C | C | A | | | |
| ZJ01 | T | C | A | C | G | A | G | C | T | T | A |
| SIN2679 | T | A | T | A | | C | C | A | | | |
| BJ01 | T | A | T | A | | C | C | A | | | |
| BJ03 | T | A | T | A | | C | C | A | | | |
| BJ02 | T | A | T | A | | C | C | A | | | |
| BJ04 | T | A | T | A | | C | C | A | | | |
| CUHK-W1 | T | A | T | A | | C | C | A | | | |
| GZ01 | C | A | T | A | | C | C | A | | | |
| GZ02102003 | T | A | T | A | | C | C | A | | | |

| | | | | | | | | | | |
|---|---|---|---|---|---|---|---|---|---|---|
| 13499 | 13500 | 14043 | 14997 | 15540 | 15573 | 16628 | 17137 | 17499 | 17540 | 17570 |

| | | | | | | | | | | | |
|---|---|---|---|---|---|---|---|---|---|---|---|
| TOR2 | T | T | T | A | A | | C | T | C | T | T |
| CUHK-SU10 | T | T | T | A | A | | C | T | C | T | T |
| SIN2748 | T | T | T | A | A | | C | T | C | T | T |
| SIN2500 | T | T | T | A | A | | C | T | C | T | T |
| HKU-39849 | A | G | T | A | A | | C | T | C | T | T |
| Urbani | T | T | T | A | A | | T | T | C | T | T |
| TW1 | T | T | T | A | A | | C | T | C | T | T |
| SIN2677 | T | T | T | A | A | | C | T | C | T | T |
| SIN2774 | T | T | T | A | A | | C | T | C | T | T |
| ZJ01 | T | T | A | T | — | T | C | T | T | A | T |
| SIN2679 | T | T | T | A | A | | C | T | C | T | T |
| BJ01 | T | T | T | A | A | | C | T | C | T | G |
| BJ03 | T | T | T | A | A | | C | T | C | T | G |
| BJ02 | T | T | T | A | A | | C | T | C | T | G |
| BJ04 | T | T | T | A | A | | C | T | C | T | G |
| CUHK-W1 | T | T | T | A | A | | C | T | C | T | G |
| GZ01 | T | T | T | A | A | | C | C | C | T | G |
| GZ02102003 | T | T | T | A | A | | C | C | C | T | G |

| | | | | | | | | | | |
|---|---|---|---|---|---|---|---|---|---|---|
| 17804 | 17852 | 18070 | 18182 | 18288 | 18971 | 19070 | 19090 | 19844 | 20369 | 20454 |

| | | | | | | | | | | | |
|---|---|---|---|---|---|---|---|---|---|---|---|
| TOR2 | C | C | G | C | C | T | A | C | A | G | G |
| CUHK-SU10 | C | T | G | C | C | T | A | C | A | G | G |
| SIN2748 | C | C | G | C | C | T | A | T | A | G | G |
| SIN2500 | C | C | G | C | C | T | A | T | A | G | G |
| HKU-39849 | C | C | A | C | C | T | A | C | A | G | G |
| Urbani | C | C | G | C | C | T | G | C | A | G | G |
| TW1 | C | C | G | C | C | T | A | C | A | G | G |
| SIN2677 | C | C | G | C | C | T | A | T | A | G | G |
| SIN2774 | C | C | G | C | C | A | A | T | A | G | G |
| ZJ01 | C | C | G | C | C | T | A | C | A | G | G |
| SIN2679 | C | C | G | C | A | T | A | C | A | G | G |
| BJ01 | C | C | G | C | C | T | A | C | G | G | G |
| BJ03 | C | C | G | C | C | T | A | C | G | T | G |
| BJ02 | T | C | G | C | C | T | A | C | G | G | G |
| BJ04 | C | C | G | C | C | T | A | C | G | G | G |
| CUHK-W1 | C | T | G | C | C | T | G | C | A | G | G |
| GZ01 | C | C | G | T | C | T | A | C | G | G | A |
| GZ02102003 | C | C | G | C | C | T | A | C | A | G | G |

| | | | | | | | | | | |
|---|---|---|---|---|---|---|---|---|---|---|
| 20462 | 20710 | 20787 | 20846 | 20854 | 20864 | 20900 | 20975 | 20998 | 20178 | 21245 |

| | | | | | | | | | | | |
|---|---|---|---|---|---|---|---|---|---|---|---|
| TOR2 | T | G | A | G | A | A | C | T | G | G | A |
| CUHK-SU10 | T | G | A | G | A | A | C | T | G | G | A |
| SIN2748 | T | N | A | G | A | A | C | T | G | G | A |
| SIN2500 | T | G | A | G | A | A | C | T | G | G | A |
| HKU-39849 | T | G | A | G | A | A | C | T | G | G | A |
| Urbani | T | G | A | G | A | A | C | T | G | G | A |
| TW1 | T | G | A | G | A | A | C | T | G | G | A |
| SIN2677 | T | G | A | G | A | A | C | T | G | G | A |
| SIN2774 | T | G | A | G | A | A | C | T | G | G | A |
| ZJ01 | T | G | A | G | A | A | C | T | G | G | A |
| SIN2679 | T | G | A | G | A | A | C | T | G | G | A |
| BJ01 | T | G | A | G | A | A | C | T | G | G | A |
| BJ03 | T | G | A | G | A | A | C | T | G | C | A |
| BJ02 | T | G | C | G | A | C | C | T | G | G | A |
| BJ04 | T | G | A | G | C | A | C | T | G | G | C |
| CUHK-W1 | T | G | A | G | A | A | C | T | G | G | A |
| GZ01 | C | G | A | G | A | A | A | C | G | G | A |
| GZ02102003 | T | G | A | A | A | A | C | T | A | G | A |

| | | | | | | | | | | |
|---|---|---|---|---|---|---|---|---|---|---|
| 21293 | 21339 | 21485 | 21494 | 21644 | 21680 | 21727 | 21297 | 22151 | 22213 | 22228 |

| | | | | | | | | | | | |
|---|---|---|---|---|---|---|---|---|---|---|---|
| TOR2 | C | A | C | G | A | A | G | A | T | C | T |
| CUHK-SU10 | C | A | C | G | A | A | G | A | T | C | T |
| SIN2748 | C | A | C | G | A | A | G | A | T | C | T |
| SIN2500 | C | A | C | G | A | A | G | A | T | C | T |
| HKU-39849 | C | A | C | G | A | A | G | A | T | C | T |
| Urbani | C | A | C | G | A | A | G | A | T | C | T |
| TW1 | C | A | C | G | A | A | G | A | T | C | T |
| SIN2677 | C | A | C | G | A | A | G | A | T | C | T |
| SIN2774 | C | A | C | G | A | A | G | A | T | C | T |
| ZJ01 | C | A | C | G | A | A | G | A | T | C | T |
| SIN2679 | C | A | C | G | A | A | G | A | T | C | T |
| BJ01 | C | A | C | G | A | A | A | A | T | C | C |
| BJ03 | C | A | C | G | A | A | A | C | T | C | C |
| BJ02 | C | A | C | A | C | A | A | A | T | C | C |
| BJ04 | C | C | C | G | A | C | G | A | T | C | C |
| CUHK-W1 | C | A | C | G | A | A | A | A | T | C | C |
| GZ01 | G | A | T | G | A | A | A | A | C | T | C |
| GZ02102003 | C | A | T | G | A | A | A | A | C | T | C |

| | | | | | | | | | | |
|---|---|---|---|---|---|---|---|---|---|---|
| 22428 | 22523 | 22528 | 22595 | 22999 | 23180 | 23226 | 23798 | 23829 | 23877 | 24075 |

| | | | | | | | | | | | |
|---|---|---|---|---|---|---|---|---|---|---|---|
| TOR2 | G | A | A | C | T | C | G | C | T | C | G |
| CUHK-SU10 | G | A | A | C | T | C | T | C | T | C | G |
| SIN2748 | G | A | A | C | T | C | T | C | T | C | G |
| SIN2500 | G | A | A | C | T | C | T | C | T | C | G |
| HKU-39849 | G | A | A | C | T | C | T | C | T | C | G |
| Urbani | G | A | A | C | T | C | T | C | T | C | G |
| TW1 | G | A | A | C | T | C | T | C | T | C | G |
| SIN2677 | G | A | A | C | T | C | T | C | T | C | G |
| SIN2774 | G | A | A | C | T | C | T | T | T | C | G |
| ZJ01 | G | A | A | T | T | C | T | C | T | C | G |
| SIN2679 | G | A | A | C | T | T | T | C | T | C | G |
| BJ01 | G | A | A | C | T | C | T | C | T | C | G |
| BJ03 | G | A | A | C | T | C | T | C | T | C | C |
| BJ02 | A | G | A | C | T | C | T | C | T | C | G |
| BJ04 | G | A | A | C | T | C | T | C | T | C | G |
| CUHK-W1 | G | A | A | C | T | C | T | C | T | C | G |
| GZ01 | A | G | G | C | A | C | T | C | G | T | G |
| GZ02102003 | A | G | G | C | T | C | T | C | G | C | G |

| | | | | | | | | | | |
|---|---|---|---|---|---|---|---|---|---|---|
| 24078 | 24499 | 24572 | 24878 | 24984 | 25304 | 25305 | 25575 | 25679 | 25785 | 25850 |

| | | | | | | | | | | | |
|---|---|---|---|---|---|---|---|---|---|---|---|
| TOR2 | A | G | T | T | A | A | G | T | A | A | A |
| CUHK-SU10 | A | G | T | T | A | G | G | T | A | A | A |
| SIN2748 | A | G | T | T | A | G | G | T | A | A | A |
| SIN2500 | A | G | T | T | A | G | G | T | A | A | A |
| HKU-39849 | A | G | T | T | A | G | G | A | A | A | A |
| Urbani | A | G | T | C | A | G | G | T | A | A | A |
| TW1 | A | G | T | T | A | G | G | T | A | A | A |
| SIN2677 | A | G | T | T | A | G | G | T | A | A | A |
| SIN2774 | A | G | T | T | A | G | G | T | A | A | A |
| ZJ01 | A | G | T | T | A | G | G | T | A | A | A |
| SIN2679 | A | G | T | T | A | G | G | T | A | A | A |
| BJ01 | A | G | T | T | A | G | G | T | C | A | A |
| BJ03 | C | G | T | T | A | G | A | T | A | A | A |
| BJ02 | A | G | T | T | A | G | A | T | A | A | A |
| BJ04 | A | T | T | T | A | G | G | T | A | A | A |
| CUHK-W1 | A | G | T | T | A | G | G | T | A | A | A |
| GZ01 | A | G | C | T | A | G | G | T | A | C | T |
| GZ02102003 | A | G | C | T | G | G | G | T | A | C | T |

| | | | | | | | | | | |
|---|---|---|---|---|---|---|---|---|---|---|
| 25990 | 26038 | 26056 | 26192 | 26434 | 26483 | 26592 | 26606 | 26863 | 27117 | 27249 |

| | | | | | | | | | | | |
|---|---|---|---|---|---|---|---|---|---|---|---|
| TOR2 | C | T | A | G | G | T | T | C | T | A | C |
| CUHK-SU10 | C | T | A | G | G | G | T | C | T | A | C |
| SIN2748 | C | T | A | G | G | T | T | C | T | A | C |
| SIN2500 | C | T | A | G | A | T | T | C | T | A | C |
| HKU-39849 | C | T | A | G | G | T | T | T | T | A | C |
| Urbani | C | T | A | G | G | T | T | C | C | A | C |
| TW1 | C | T | A | G | G | T | T | C | T | A | C |
| SIN2677 | C | T | A | G | G | T | T | C | T | G | C |
| SIN2774 | C | T | A | G | G | T | T | C | T | A | C |
| ZJ01 | C | T | A | G | G | T | T | C | T | A | C |
| SIN2679 | C | T | A | G | G | T | T | C | T | A | C |
| BJ01 | C | T | C | G | G | T | T | C | T | A | T |
| BJ03 | C | T | C | G | G | T | T | C | T | A | T |
| BJ02 | T | T | A | G | G | T | T | C | T | A | T |
| BJ04 | C | T | A | G | G | T | T | C | T | A | T |
| CUHK-W1 | C | T | A | G | G | T | T | C | T | A | C |
| GZ01 | C | A | A | A | G | T | T | C | T | A | T |
| GZ02102003 | C | A | A | G | G | T | C | C | T | A | C |

| | | | | | |
|---|---|---|---|---|---|
| 27749 | 27789-27944 | 27817-27821 | 27834 | 27891-27919 | 28125 |

| | | | | | | |
|---|---|---|---|---|---|---|
| TOR2 | | AAACTT | CTCTA | T | | C |
| CUHK-SU10 | | AAACTT | CTCTA | T | | C |
| SIN2748 | | AAACTT | — | T | | C |
| SIN2500 | | AAACTT | CTCTA | T | | C |
| HKU-39849 | | AAACTT | CTCTA | T | | C |
| Urbani | | AAACTT | CTCTA | T | | C |
| TW1 | | AAACTT | CTCTA | T | | C |
| SIN2677 | | — | CTCTA | T | | C |
| SIN2774 | | AAACTT | CTCTA | T | | C |
| ZJ01 | A | AAACTT | CTCTA | T | | C |
| SIN2679 | | AAACTT | CTCTA | T | | C |
| BJ01 | | AAACTT | CTCTA | C | | C |
| BJ03 | | AAACTT | CTCTA | C | | C |
| BJ02 | | AAACTT | CTCTA | C | | C |
| BJ04 | | AAACTT | CTCTA | C | | C |
| CUHK-W1 | | AAACTT | CTCTA | C | | C |
| GZ01 | | AAACTT | CTCTA | C | CCTACTGGTTACCAACCTGAATGGAATAT | T |
| GZ02102003 | | AAACTT | CTCTA | C | CCTACTGGTTACCAACCTGAATGGAATAT | T |

| | | | | | |
|---|---|---|---|---|---|
| 28509 | 28574 | 28575 | 28615 | 28732 | 29283 |

| | | | | | | |
|---|---|---|---|---|---|---|
| TOR2 | A | T | G | A | G | G |
| CUHK-SU10 | A | T | G | A | T | G |
| SIN2748 | A | T | G | A | G | G |
| SIN2500 | A | T | G | A | G | G |
| HKU-39849 | A | T | G | A | G | G |
| Urbani | A | T | G | A | G | G |
| TW1 | A | T | G | A | G | G |
| SIN2677 | A | T | G | A | G | G |
| SIN2774 | A | G | A | A | G | G |
| ZJ01 | — | T | G | A | G | G |
| SIN2679 | A | T | G | A | G | G |
| BJ01 | A | T | G | A | G | G |
| BJ03 | A | T | G | T | G | G |
| BJ02 | A | T | G | A | G | G |
| BJ04 | A | T | G | A | G | G |
| CUHK-W1 | A | T | G | A | G | G |
| GZ01 | A | T | G | A | G | G |
| GZ02102003 | A | T | G | A | G | A |

| | | | | | | | | | | | | | | | |
|---|---|---|---|---|---|---|---|---|---|---|---|---|---|---|---|
| 509 | 652 | 937 | 1180 | 1206 | 1476 | 2601 | 3165 | 3274 | 3326 | 3626 | 4220 | 4250 | 4876 | 4952 | 5251 |

| | | | | | | | | | | | | | | | | |
|---|---|---|---|---|---|---|---|---|---|---|---|---|---|---|---|---|
| TOR2 | G | G | A | G | T | A | T | A | A | T | T | A | T | T | T | C |
| CUHK-SU10 | G | G | A | G | T | A | T | A | A | T | T | A | T | T | T | C |
| SIN2748 | G | G | A | G | T | A | T | A | A | T | T | A | T | T | T | C |
| SIN2500 | G | G | A | G | T | A | T | A | A | T | T | A | T | T | T | C |
| HKU-39849 | G | G | A | G | T | A | C | A | A | T | T | A | T | T | T | C |
| Urbani | G | G | A | G | T | A | T | A | A | T | T | A | T | T | T | C |
| TW1 | G | G | A | G | T | A | T | G | A | T | T | A | T | T | T | C |
| SIN2677 | G | G | A | G | T | G | T | A | A | T | T | A | T | T | T | C |
| SIN2774 | G | G | A | G | T | A | T | A | A | T | T | A | T | T | T | C |
| SIN2679 | G | G | A | G | T | A | T | A | A | T | T | A | T | T | T | C |
| BJ01 | G | G | A | G | T | A | T | A | A | T | T | A | T | T | T | C |
| BJ03 | G | G | A | G | T | A | T | A | C | T | T | A | T | T | T | C |
| BJ02 | G | G | A | G | T | A | T | A | A | T | T | A | T | T | A | C |
| BJ04 | G | G | A | T | T | A | T | A | A | T | T | A | T | T | T | C |
| CUHK-W1 | G | G | A | G | T | A | T | A | A | T | T | A | T | T | T | C |
| GZ01 | T | A | C | G | T | A | T | A | A | T | C | G | C | A | T | C |
| GZ02102003 | T | G | A | G | C | A | T | A | A | C | C | G | T | T | T | A |
| A change | G-C | G-R | K-Q | V-F | | | | | N-H | V-A | * | K-R | F-S | S-T | M-K | L-T |
| ORF | 1a | 1a | 1a | 1a | 1a | 1a | 1a | 1a | 1a | 1a | 1a | 1a | 1a | 1a | 1a | 1a |

| | | | | | | | | | | | | | | |
|---|---|---|---|---|---|---|---|---|---|---|---|---|---|---|
| 5427 | 5548 | 5591 | 5394 | 5681 | 6148 | 6612 | 6929 | 7746 | 7919 | 7930 | 7954 | 8387 | 8417 | 8502 |

| | | | | | | | | | | | | | | | |
|---|---|---|---|---|---|---|---|---|---|---|---|---|---|---|---|
| TOR2 | A | A | A | A | G | T | G | G | G | C | G | T | G | G | T |
| CUHK-SU10 | A | A | A | A | G | T | G | G | G | C | G | T | G | G | T |
| SIN2748 | A | A | A | A | G | T | G | G | G | C | G | T | G | G | T |
| SIN2500 | A | A | A | A | G | T | G | G | G | C | G | T | G | G | T |
| HKU-39849 | A | A | A | A | G | T | G | G | G | C | A | T | C | C | T |
| Urbani | A | A | A | A | G | T | G | G | G | T | G | T | G | G | T |
| TW1 | A | A | A | A | G | T | G | G | G | C | G | T | G | G | T |
| SIN2677 | A | A | A | A | G | T | G | G | G | C | G | T | G | G | T |
| SIN2774 | A | A | A | A | G | T | G | G | G | C | G | T | G | G | T |
| SIN2679 | A | A | A | A | G | T | G | G | G | C | G | T | G | G | T |
| BJ01 | A | A | A | A | G | T | G | G | G | C | G | T | G | G | T |
| BJ03 | C | C | C | C | G | T | G | G | G | C | G | T | G | G | T |
| BJ02 | A | A | A | A | T | T | G | G | G | C | G | T | G | G | T |
| BJ04 | A | A | A | A | G | A | G | G | G | C | G | T | G | G | T |
| CUHK-W1 | A | A | A | A | G | T | G | G | T | C | G | T | G | G | T |
| GZ01 | A | A | A | A | G | T | T | A | G | C | G | C | G | G | T |
| GZ02102003 | A | A | A | A | G | T | T | A | G | C | G | T | G | G | G |
| A change | | T-L | Q-P | Q-P | G-V | L-T | *L-F | C-V | | A-V | D-N | S-P | S-T | R-T | *C-W |
| ORF | 1a | 1a | 1a | 1a | 1a | 1a | 1a | 1a | 1a | 1a | 1a | 1a | 1a | 1a | 1a |

| | | | | | | | | | | | | |
|---|---|---|---|---|---|---|---|---|---|---|---|---|
| 8559 | 8573 | 8816 | 8947 | 9096 | 9177 | 9405 | 9480 | 9855 | 10030 | 10324 | 10551 | 10588 |

| | | | | | | | | | | | | | |
|---|---|---|---|---|---|---|---|---|---|---|---|---|---|
| TOR2 | T | G | C | T | C | T | T | T | C | G | A | A | A |
| CUHK-SU10 | T | G | C | T | C | T | T | T | C | G | A | A | A |
| SIN2748 | T | G | C | T | C | T | T | T | C | G | A | A | A |
| SIN2500 | T | G | C | T | C | T | T | T | C | G | A | A | A |
| HKU-39849 | T | G | C | T | C | T | T | T | C | G | A | A | A |
| Urbani | T | G | C | T | C | T | T | T | C | G | A | A | A |
| TW1 | T | G | C | T | C | T | T | T | C | G | A | A | A |
| SIN2677 | T | G | C | T | C | T | T | T | C | G | A | A | A |
| SIN2774 | T | G | C | T | C | T | T | T | C | G | A | A | A |
| SIN2679 | T | G | C | T | C | T | T | T | C | G | A | A | A |
| BJ01 | T | T | C | T | C | T | C | T | T | G | A | A | C |
| BJ03 | T | G | C | T | C | T | C | T | T | G | A | A | A |
| BJ02 | T | T | C | T | C | T | C | T | T | G | A | G | A |
| BJ04 | T | G | C | T | C | T | T | T | T | G | A | A | A |
| CUHK-W1 | T | G | C | T | C | T | C | C | C | G | A | A | A |
| GZ01 | C | G | T | A | T | C | C | C | C | A | G | A | C |
| GZ02102003 | C | G | T | A | T | C | C | C | C | A | A | A | A |
| A change | | V-L | * | | * | V-A | V-A | V-A | A-V | | | Q-P | |
| ORF | 1a | 1a | 1a | 1a | 1a | 1a | 1a | 1a | 1a | 1a | 1a | 1a | 1a |

| | | | | | | | | | | |
|---|---|---|---|---|---|---|---|---|---|---|
| 10729 | 11718 | 11972 | 11975 | 12151 | 13499 | 13500 | 16628 | 17137 | 17570 | 17804 |

| | | | | | | | | | | | |
|---|---|---|---|---|---|---|---|---|---|---|---|
| TOR2 | C | A | G | A | T | T | T | C | T | T | C |
| CUHK-SU10 | C | A | G | A | T | T | T | C | T | T | C |
| SIN2748 | C | A | G | A | T | T | T | C | T | T | C |
| SIN2500 | C | A | G | A | T | T | T | C | T | T | C |
| HKU-39849 | C | A | G | A | T | A | G | C | T | T | C |
| Urbani | C | A | G | A | T | T | T | T | T | T | C |
| TW1 | C | A | G | A | T | T | T | C | T | T | C |
| SIN2677 | C | A | G | A | T | T | T | C | T | T | C |
| SIN2774 | C | A | G | A | T | T | T | C | T | T | C |
| SIN2679 | C | A | G | A | T | T | T | C | T | T | C |
| BJ01 | C | A | G | A | T | T | T | C | T | G | C |
| BJ03 | C | A | A | A | T | T | T | C | T | G | C |
| BJ02 | C | A | G | T | T | T | T | C | T | G | T |
| BJ04 | A | C | G | A | T | T | T | C | T | G | C |
| CUHK-W1 | C | A | G | A | T | T | T | C | T | G | C |
| GZ01 | C | A | G | A | C | T | T | C | C | G | C |
| GZ02102003 | C | A | G | A | T | T | T | C | C | G | C |
| A change | *D-E | N-T | D-N | T-F | | | V-S | * | L-S | D-E | |
| ORF | 1a | 1a | 1a | 1a | 1a | 1b | 1b | 1b | 1b | 1b | 1b |

| | | | | | | | | | | |
|---|---|---|---|---|---|---|---|---|---|---|
| 17852 | 18071 | 18182 | 18288 | 18971 | 19070 | 19090 | 19844 | 20369 | 20454 | 20462 |

| | | | | | | | | | | | |
|---|---|---|---|---|---|---|---|---|---|---|---|
| TOR2 | C | G | C | C | T | A | C | A | G | G | T |
| CUHK-SU10 | T | G | C | C | T | A | C | A | G | G | T |
| SIN2748 | C | G | C | C | T | A | T | A | G | G | T |
| SIN2500 | C | G | C | C | T | A | T | A | G | G | T |
| HKU-39849 | C | A | C | C | T | A | C | A | G | G | T |
| Urbani | C | G | C | C | T | G | C | A | G | G | T |
| TW1 | C | G | C | C | T | A | C | A | G | G | T |
| SIN2677 | C | G | C | C | T | A | T | A | G | G | T |
| SIN2774 | C | G | C | C | A | A | T | A | G | G | T |
| SIN2679 | C | G | C | A | T | A | C | A | G | G | T |
| BJ01 | C | G | C | C | T | A | C | G | G | G | T |
| BJ03 | C | G | C | C | T | A | C | G | T | G | T |
| BJ02 | C | G | C | C | T | A | C | G | G | G | T |
| BJ04 | C | G | C | C | T | A | C | G | G | G | T |
| CUHK-W1 | T | G | C | C | T | G | C | A | G | G | T |
| GZ01 | C | G | T | C | T | A | C | G | G | A | C |
| GZ02102003 | C | G | C | C | T | A | C | A | G | G | T |
| A change | | | | L-T | | | T-I | | *M-T | D-N | |
| ORF | 1b | 1b | 1b | 1b | 1b | 1b | 1b | 1b | 1b | 1b | 1b |

| | | | | | | | | | | |
|---|---|---|---|---|---|---|---|---|---|---|
| 20710 | 20787 | 20846 | 20854 | 20864 | 20900 | 20975 | 20998 | 21078 | 21245 | 21293 |

| | | | | | | | | | | | |
|---|---|---|---|---|---|---|---|---|---|---|---|
| TOR2 | G | A | G | A | A | C | T | G | G | A | C |
| CUHK-SU10 | G | A | G | A | A | C | T | G | G | A | C |
| SIN2748 | N | A | G | A | A | C | T | G | G | A | C |
| SIN2500 | G | A | G | A | A | C | T | G | G | A | C |
| HKU-39849 | G | A | G | A | A | C | T | G | G | A | C |
| Urbani | G | A | G | A | A | C | T | G | G | A | C |
| TW1 | G | A | G | A | A | C | T | G | G | A | C |
| SIN2677 | G | A | G | A | A | C | T | G | G | A | C |
| SIN2774 | G | A | G | A | A | C | T | G | G | A | C |
| SIN2679 | G | A | G | A | A | C | T | G | G | A | C |
| BJ01 | G | A | G | A | A | C | T | G | G | A | C |
| BJ03 | G | A | G | A | A | C | T | G | C | A | C |
| BJ02 | G | C | G | A | C | C | T | G | G | A | C |
| BJ04 | G | A | G | C | A | C | T | G | G | C | C |
| CUHK-W1 | G | A | G | A | A | C | T | G | G | A | C |
| GZ01 | G | A | G | A | A | A | C | G | G | A | G |
| GZ02102003 | G | A | A | A | A | C | T | A | G | A | C |
| A change | ? | M-L | | Q-P | | *D-E | | R-K | A-P | *D-E | *N-K |
| ORF | 1b | 1b | 1b | 1b | 1b | 1b | 1b | 1b | 1b | 1b | 1b |

| | | | | | | | | | | |
|---|---|---|---|---|---|---|---|---|---|---|
| 21339 | 21485 | 21494 | 21644 | 21680 | 21727 | 21927 | 22151 | 22213 | 22228 | 22428 |

| | | | | | | | | | | | |
|---|---|---|---|---|---|---|---|---|---|---|---|
| TOR2 | A | C | G | A | A | G | A | T | C | T | G |
| CUHK-SU10 | A | C | G | A | A | G | A | T | C | T | G |
| SIN2748 | A | C | G | A | A | G | A | T | C | T | G |
| SIN2500 | A | C | G | A | A | G | A | T | C | T | G |
| HKU-39849 | A | C | G | A | A | G | A | T | C | T | G |
| Urbani | A | C | G | A | A | G | A | T | C | T | G |
| TW1 | A | C | G | A | A | G | A | T | C | T | G |
| SIN2677 | A | C | G | A | A | G | A | T | C | T | G |
| SIN2774 | A | C | G | A | A | G | A | T | C | T | G |
| SIN2679 | A | C | G | A | A | G | A | T | C | T | G |
| BJ01 | A | C | G | A | A | A | A | T | C | C | G |
| BJ03 | A | C | G | A | A | A | C | T | C | C | G |
| BJ02 | A | C | A | C | A | A | A | T | C | C | A |
| BJ04 | C | C | G | A | C | G | A | T | C | C | G |
| CUHK-W1 | A | C | G | A | A | A | A | T | C | C | G |
| GZ01 | A | T | G | A | A | A | A | C | T | C | A |
| GZ02102003 | A | T | G | A | A | A | A | C | T | C | A |
| A change | K-Q | * | | | | G-D | M-L | | S-L | I-T | G-R |
| ORF | 1b | S | S | S | S | S | S | S | S | S | S |

| | | | | | | | | | | |
|---|---|---|---|---|---|---|---|---|---|---|
| 22523 | 22528 | 22999 | 23180 | 23226 | 23798 | 23829 | 23877 | 24075 | 24078 | 24499 |

| | | | | | | | | | | | |
|---|---|---|---|---|---|---|---|---|---|---|---|
| TOR2 | A | A | T | C | G | C | T | C | G | A | G |
| CUHK-SU10 | A | A | T | C | T | C | T | C | G | A | G |
| SIN2748 | A | A | T | C | T | C | T | C | G | A | G |
| SIN2500 | A | A | T | C | T | C | T | C | G | A | G |
| HKU-39849 | A | A | T | C | T | C | T | C | G | A | G |
| Urbani | A | A | T | C | T | C | T | C | G | A | G |
| TW1 | A | A | T | C | T | C | T | C | G | A | G |
| SIN2677 | A | A | T | C | T | C | T | C | G | A | G |
| SIN2774 | A | A | T | C | T | T | T | C | G | A | G |
| SIN2679 | A | A | T | T | T | C | T | C | G | A | G |
| BJ01 | A | A | T | C | T | C | T | C | G | A | G |
| BJ03 | A | A | T | C | T | C | T | C | C | C | G |
| BJ02 | G | A | T | C | T | C | T | C | G | A | G |
| BJ04 | A | A | T | C | T | C | T | C | G | A | T |
| CUHK-W1 | A | A | T | C | T | C | T | C | G | A | G |
| GZ01 | G | G | A | C | T | C | G | T | G | A | G |
| GZ02102003 | G | G | T | C | T | C | G | C | G | A | G |
| A change | | K-R | F-Y | | S-A | | Y-D | P-S | V-L | S-R | R-M |
| ORF | S | S | S | S | S | S | S | S | S | S | S |

| | | | | | | | | | | |
|---|---|---|---|---|---|---|---|---|---|---|
| 24572 | 24878 | 24984 | 25304 | 25305 | 25575 | 25679 | 25785 | 25850 | 25990 | 26038 |

| | | | | | | | | | | | |
|---|---|---|---|---|---|---|---|---|---|---|---|
| TOR2 | T | T | A | A | G | T | A | A | A | C | T |
| CUHK-SU10 | T | T | A | G | G | T | A | A | A | C | T |
| SIN2748 | T | T | A | G | G | T | A | A | A | C | T |
| SIN2500 | T | T | A | G | G | T | A | A | A | C | T |
| HKU-39849 | T | T | A | G | G | A | A | A | A | C | T |
| Urbani | T | C | A | G | G | T | A | A | A | C | T |
| TW1 | T | T | A | G | G | T | A | A | A | C | T |
| SIN2677 | T | T | A | G | G | T | A | A | A | C | T |
| SIN2774 | T | T | A | G | G | T | A | A | A | C | T |
| SIN2679 | T | T | A | G | G | T | A | A | A | C | T |
| BJ01 | T | T | A | G | G | T | C | A | A | C | T |
| BJ03 | T | T | A | G | A | T | A | A | A | C | T |
| BJ02 | T | T | A | G | A | T | A | A | A | T | T |
| BJ04 | T | T | A | G | G | T | A | A | A | C | T |
| CUHK-W1 | T | T | A | G | G | T | A | A | A | C | T |
| GZ01 | C | T | A | G | G | T | A | C | T | C | A |
| GZ02102003 | C | T | G | G | G | T | A | C | T | C | A |
| A change | | | K-E | G-R | G-E | M-K | K-Q | E-A | R-W | T-I | L-Q |
| ORF | S | S | S | orf3 | orf3 | orf3 | orf3 | orf3 | orf3 | orf3 | orf3 |

| | | | | | | | | | | |
|---|---|---|---|---|---|---|---|---|---|---|
| 26056 | 26192 | 26434 | 26483 | 26592 | 26606 | 26863 | 27117 | 27249 | 27782-7 | 22781-4 |

| | | | | | | | | | | | |
|---|---|---|---|---|---|---|---|---|---|---|---|
| TOR2 | A | G | G | T | T | C | T | A | C | AAACTT | CTCTA |
| CUHK-SU10 | A | G | G | G | T | C | T | A | C | AAACTT | CTCTA |
| SIN2748 | A | G | G | T | T | C | T | A | C | AAACTT | — |
| SIN2500 | A | G | A | T | T | C | T | A | C | AAACTT | CTCTA |
| HKU-39849 | A | G | G | T | T | T | T | A | C | AAACTT | CTCTA |
| Urbani | A | G | G | T | T | C | C | A | C | AAACTT | CTCTA |
| TW1 | A | G | G | T | T | C | T | A | C | AAACTT | CTCTA |
| SIN2677 | A | G | G | T | T | C | T | G | C | — | CTCTA |
| SIN2774 | A | G | G | T | T | C | T | A | C | AAACTT | CTCTA |
| SIN2679 | A | G | G | T | T | C | T | A | C | AAACTT | CTCTA |
| BJ01 | C | G | G | T | T | C | T | A | T | AAACTT | CTCTA |
| BJ03 | C | G | G | T | T | C | T | A | T | AAACTT | CTCTA |
| BJ02 | A | G | G | T | T | C | T | A | T | AAACTT | CTCTA |
| BJ04 | A | G | G | T | T | C | T | A | T | AAACTT | CTCTA |
| CUHK-W1 | A | G | G | T | T | C | T | A | C | AAACTT | CTCTA |
| GZ01 | A | A | G | T | T | C | T | A | T | AAACTT | CTCTA |
| GZ02102003 | A | G | G | T | C | C | T | A | C | AAACTT | CTCTA |
| A change | Q-P | V-M | | F-C | | A-V | S-P | E-G | P-L | [symbol] | [symbol] |
| ORF | orf3 | E | M | M | M | M | M | orf7 | orf7 | orf10 | orf10 |

| | | | | | |
|---|---|---|---|---|---|
| 27834 | 27891-27919 | 28125 | 28615 | 28732 | 29283 |

| | | | | | | |
|---|---|---|---|---|---|---|
| TOR2 | T | | C | A | G | G |
| CUHK-SU10 | T | | C | A | T | G |
| SIN2748 | T | | C | A | G | G |
| SIN2500 | T | | C | A | G | G |
| HKU-39849 | T | | C | A | G | G |
| Urbani | T | | C | A | G | G |
| TW1 | T | | C | A | G | G |
| SIN2677 | T | | C | A | G | G |
| SIN2774 | T | | C | A | G | G |
| SIN2679 | T | | C | A | G | G |
| BJ01 | C | | C | A | G | G |
| BJ03 | C | | C | T | G | G |
| BJ02 | C | | C | A | G | G |
| BJ04 | C | | C | A | G | G |
| CUHK-W1 | C | | C | A | G | G |
| GZ01 | C | CCTACTGGTTACCAACCTGAATGGAATAT | T | A | G | G |
| GZ02102003 | C | CCTACTGGTTACCAACCTGAATGGAATAT | T | A | G | A |
| A change | C-R | [symbol] | | N-Y | G-C | |
| ORF | orf10 | orf10 - orf11 | N | ORF14 | ORF14 | |

Note:
only non-homologous variation was shown. The position of each nucleotide was shown based on TOR2 SARS-CoV. The substitution of amino acid, related proteins and open reading frames were also indicated.

An important result from the sequencing analysis was that the coding of amino acids of ORF10 and ORF11 were changed by the special twenty nine bases in GZ02102003. The results were shown in FIGS. 2A and 2B.

The specialty of the methods in the invention was that the total cDNA was transcribed directly from the infected pulmonary tissue of the body, and the full sequence of the SARS coronavirus was tested using SNP sequencing.

The most important discovery of the invention was the special 29 nucleotides sequence fragment (CCTACTGGTTACCAACCTGAATGGAATAT, see table 1) which was obtained from the patient. This discovery indicated the following 3 important facts: 1) This sequence only existed in the earliest SARS victim tissue samples, while this sequence was missing from the SARS victims infected after March 2003 (see table 1). 2) The existence of this sequence completely changed ORF10 and ORF11 (see FIGS. 2A and 2B). 3) This sequence also existed in the SARS coronavirus isolated from the wild civet. Thus, it was believed that the SARS virus infecting human was originated from civet, based on the migration from civet to human.

### CONCISE DESCRIPTION OF THE FIGURES

FIG. 1 was the thin slice of infected pulmonary tissue observed under electron microscope.

FIG. 2A and FIG. 2B was the comparison of ORF10 and ORF11 respectively

FIG. 3 was the final product of PCR. DNA Marker: from bottom to top 1.100 bp; 2.250 bp; 3.500 bp; 4.750 bp; 5.1000 bp; 6.2000 bp; 7.2500 bp 8.5000 bp; 9.7500 bp; 10.10000 bp; 11.15000 bp. PCR fragment: from left to right S full sequence; S1 fragment; S2 fragment; E protein; M protein; N protein; PXN fragment.

FIG. 4 was plasmid pMD18-T (provided by Takara).

FIG. 5 was the pMD18-T cloning map for S1, S2, E, M, N, and X2.

FIG. 6 was the map for pcDNA3.1(+)(−).

FIG. 7 was the cloning map for pcDNA3.1 (+)(−) cut by restriction Enzyme in (S1, S2, E, M, N and X2).

FIG. 8A-8D was the immunological testing results for part of the nucleotide sequence of the invention. FIG. 8A only showed the S1 which was the adenovirus vector for S protein (spike protein). FIG. 8B showed the S2 which was the adenovirus vector for S and E proteins. FIG. 8C showed the S3G which was the adenovirus vector for S, M and E proteins. FIG. 8D showed the S3N which was the adenovirus vector for E, M and N proteins.

FIG. 9 was the immunological test results for part of the nucleotide sequence of the invention. The S3G which was the adenovirus vector for S, M and E protein, was used as vaccine. PBS was used in control.

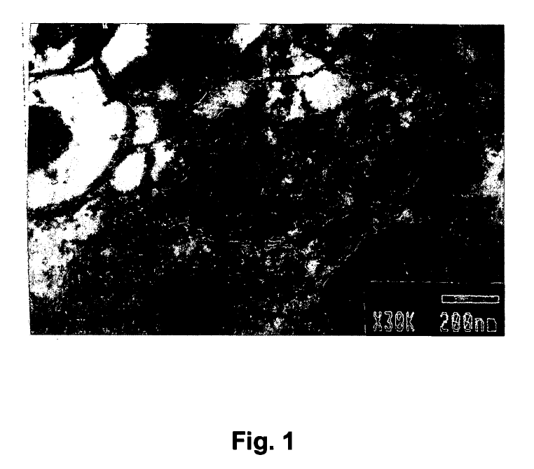

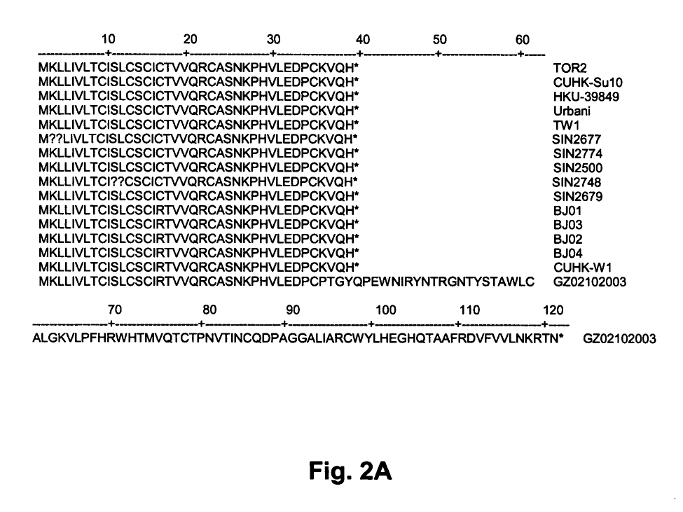

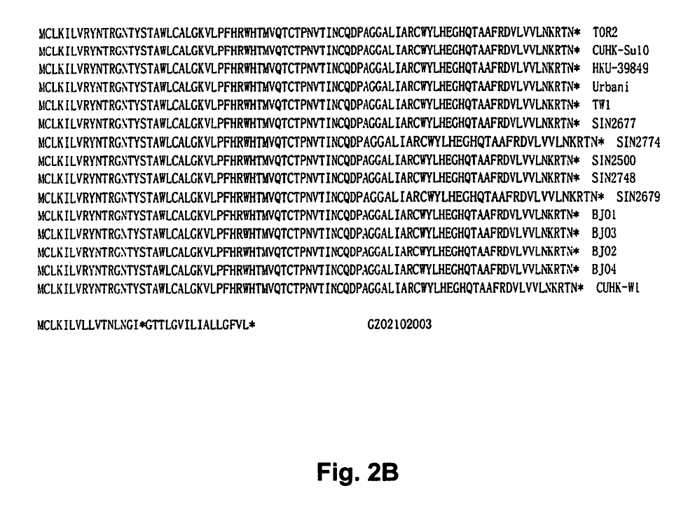

### DETAIL DESCRIPTION OF EMBODIMENTS

The embodiments of the invention were described in the followings. However, the invention was only described but not limited by those embodiments. The invention was only limited by the attached claims.

### Example 1

### Obtaining of SARS Virus Gene Fragments

Obtaining of SARS Virus RNA

1.1 Materials
1.1.2 Lung Tissue Containing SARS Virus: Obtained from a Female Guangzhou patient who died of SARS.
1.1.3 TRIZOL Reagent: purchased from GIBCOBRL, used as total RNA extraction kit.

1.2 Methods
1.2.1 100 mg of infected lung tissue was obtained from fridge of −80° C., and grounded in clean glass molar.
1.2.2 1 ml of TRIZOL was put in glass molar, gently mixed in the container with the grounded lung tissues, and collected in centrifuge tube of 1.5 ml.
1.2.3 The centrifuge tube was set in room temperature for 5 minutes. 0.2 ml of chloroform was put in the centrifuge tube. The tube was vigorously stirred and set in room temperature for 3 minutes.
1.2.4 The tube was then centrifuged at 4° C. for 15 minutes, at 12000 g/minute.
1.2.5 The supernatant containing RNA was collected after centrifugation.
   0.5 ml of isopropane was put in the collected liquid. The liquid was sat in room temperature for 15 minutes.
1.2.6 The liquid was centrifuged at 12000 g/minute at 4° C. for 10 minutes.
1.2.7 The supernatant was discarded. The RNA precipitate was washed using 75% alcohol.
1.2.8 The RNA precipitate was slightly dried in the air and was added 50 ml of aseptic water.

Production of cDNA

2.1 Materials
2.1.1 cDNA production kit: RNA PCR Kit (AMV) Ver.2.1, purchased from Bao Biotech Corp.
2.1.2 SARS RNA: extracted by the infection medicine of South Hospital.

2.2 Methods
2.2.1 Reaction Mixture

| | |
|---|---|
| MgCl | 4.0 μl |
| Buffer | 2.0 μl |
| dNTP | 2.0 μl |
| RNAase Inhibitor | 0.5 μl |
| Random Primer | 1.0 μl |
| Orligo dT primer | 1.0 μl |
| RNA template | 1.0 μl |
| Transcriptase enzyme | 1.0 μl |
| Water | 7.5 μl |

2.2.2 Reaction Procedures
   Step 1: incubation at 37° C. for 50 minutes
   Step 2: incubation at 50° C. for 2 minutes
   Step 3: incubation at 37° C. for 5 minutes
   Step 4: repeat step 2, and 3 for 5 times
   Step 5: incubation at 95° C. for 3 minutes

PCR Amplification

3.1 Materials
3.1.1 PCR kit: KaTaRa Ex Taq. From Bao Biotech Corp.
3.1.2 cDNA produced by the inventor

3.2 Methods
3.2.1 Reaction Mixture

| | |
|---|---|
| 10X Ex Taq buffer | 1.0 μl |
| dNTP mixture | 0.8 μl |
| cDNA template | 1.0 μl |
| random primer | 0.5 μl |
| random primer | 0.5 μl |
| Taq enzyme | 0.05 μl |
| Water | 6.15 μl |

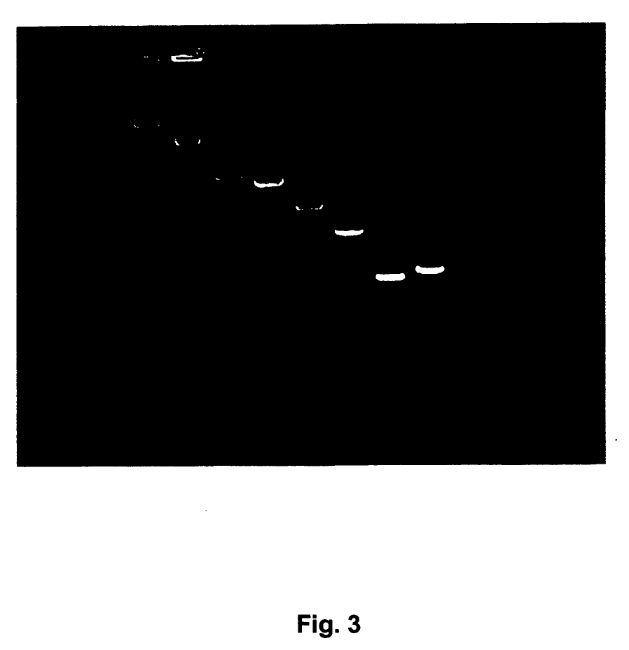

3.2.2 Reaction Procedure
   Step 1: incubation at 94° C. for 3 minutes
   Step 2: incubation at 94° C. for 30 seconds
   Step 3: incubation at 58° C. for 20 seconds
   Step 4: incubation at 72° C. for 40 seconds (note: incubation time varies from 40 seconds to 4 minutes, based on the molecule weight of the amplification fragment)
   Step 5: repeat step 2, 3 and 4 for 34 times
   Step 6: incubation at 72° C. for 5 minutes
Results were shown in FIG. 3.

### Example 2

### Cloning of the Gene Fragment which was Related to Antigen of the SARS Virus

1. PCR Amplification of 6 Antigen Gene Fragments

ATG (start codon) was included in all of the designed PCR primers. And all PCR products had stop codon at 3′ end. Thus all the fragments were effectively expressed after being cloned to its vector. Those primers were produced by Huada Gene Shanghai Dinan Biotech Ltd Corp. They were dissolved in 200 μl minipore aseptic water per OD. Then the primers were diluted in 5 times and used as 10× concentration in PCR reaction.

PCR kit used in this experiment was purchased from Takara Corp. The PCR template was pGEM T Easy clones corresponding to the clone of pGEM-T Easy. The PCR reaction conditions included: two primers of 1/10 volume, 10˜50 ng template, dNTP, 10×PCR buffer of 1/10 volume, and 2 units of Taq enzyme. All the above ingredients were added with sterile water until working volume up to 10˜25 μl. The procedures of PCR were: 94° C. for 4 minutes, 94° C. for 30 seconds, 58° C. for 30 seconds, 72° C. for 2.5 minutes for 30 cycles, and at last 72° C. for 10 minutes. All PCR reactions were implemented on PCR machine from Eppendorf Corp.

All the PCR products were shown in FIG. 3.

2. Construction of pMD18-T Cloning for 6 Antigen Gene Fragments

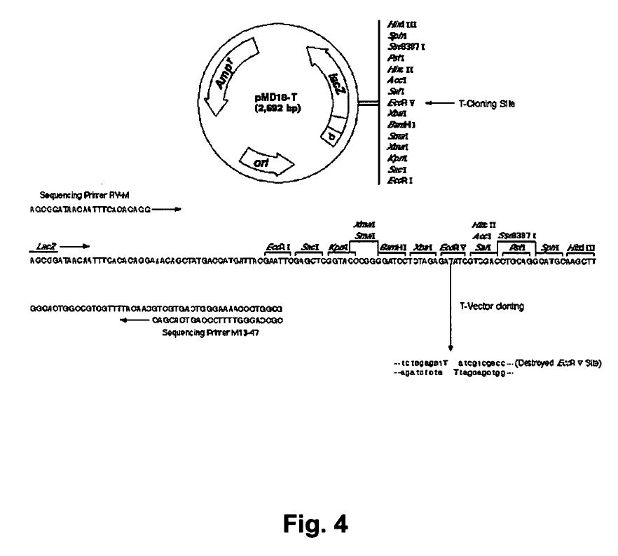

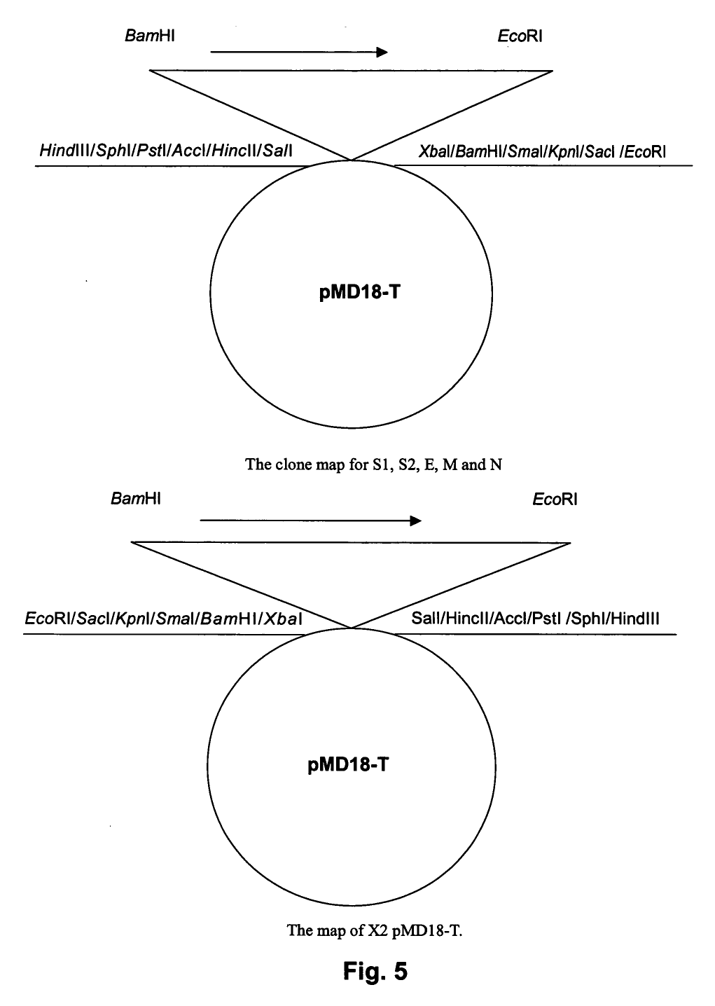

All the PCR products were purified using PCR Purification Kit from Qiagen Corp. The products were connected with pMD18-T (TA clone vector from Takara, see FIG. 4) in 2:1˜5:1 mol concentration ratio using unit complicing enzyme, the total volume is 10˜20 μl. Then the vector was transfected into DH5α sensitive cells and spread on LB medium with 100 μg/ml Ampicillin and IPTG/X-gal. Then the white cell colonies were selected from the plate and cultured in 4 ml LB with 100 μg/ml Ampicillin. Plasmids were extracted (using miniprep extraction kit from Qiagen Corp) and cut by enzymes (S1, S2, E, M and N clones were cut by BamHI and EcoRI, X2 clone was cut by KpnI and EcoRI) to detect whether the obtained clones comprise the right size of insertion fragments (See FIG. 5). The verified cloning fragments were sent for inserted fragments sequencing in Huada Gene Shanghai Dinan Biotech Ltd Corps. Thus the cloning fragments were further affirmed.

3. Construction of pcDNA3.1 Cloning for 6 Antigen Gene Fragments

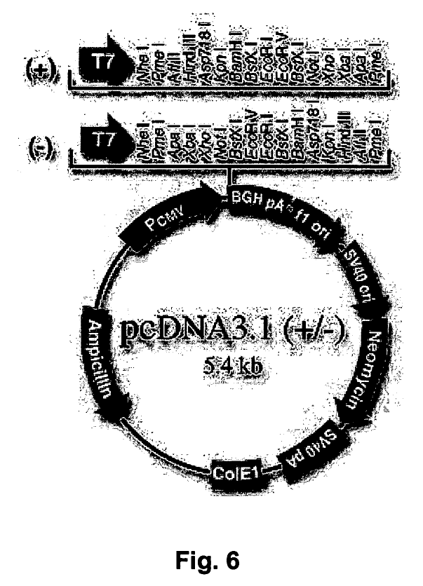

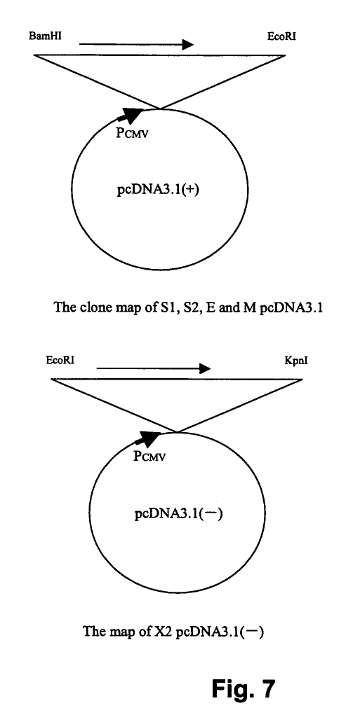

The sequence verified 5 pMD18-T cloning (containing S1, S2, E, M and N), were cut by restriction enzyme BamHI and EcoRI. Then the samples were applied in electrophoresis to separate the inserted cloning fragments from pMD18-T. The fragments S1, S2, E, M and N were purified using gel extraction kit from Qiagen. Finally, those fragments were respectively cloned to pcDNA3.1(+) vector which was already cut by BamHI and EcoRI (see FIG. 6). For X2 pMD18-T cloning, the pcDNA3.1(−) was cut by EcoRI and KpnI. Then the fragment was inserted into pcDNA3.1(−) vector. The obtained recombinant clones of pcDNA3.1(+)/(−) with S1, S2, E, M, N and X2 (see FIG. 7), were used as DNA vaccine candidates, and applied in animal experiments.

TABLE 2
PCR primers and relative PCR reation templates and its products

| PCR Product | Primer | Template |
|---|---|---|
| S1(~1980 bp) | 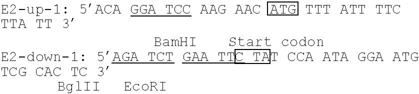 | Jin Li's #1 pGEM-TEasy Cloning |
| S2(~1940 bp) | 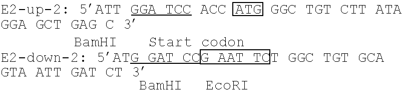 | Jin Li's #2 pGEM-TEasy Cloning |
| E(~300 bp) | 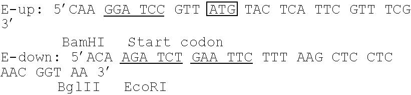 | Jin Li's #5 pGEM-TEasy Cloning |
| M(~760 bp) | 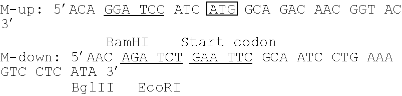 | Jin Li's #4 pGEM-TEasy Cloning |
| N(~1315 bp) | 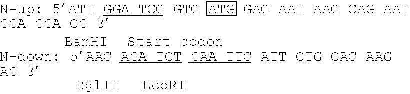 | Jin Li's #3 pGEM-TEasy Cloning |
| X2(~380 bp) | 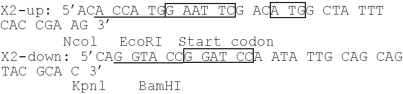 | Jin Li's #6 pGEM-TEasy Cloning |

TABLE 3
6 the design of the fragments cloning

| | | pcDNA3.1 vector used for |
|---|---|---|
| Target Clone | Origin of the inserted fragment | connection of clone |
| pcDNA3.1(+)-S1 | BamHI/EcoRI cut S1 fragment(~1980 bp), | BamHI/EcoRI cut |
| | from pMD18-T/S1 | pcDNA3.1(+) |
| pcDNA3.1(+)-S2 | BamHI/EcoRI cut S2 gragment(~1940 bp), | BamHI/EcoRI cut |
| | from pMD18-T/S2 | pcDNA3.1(+) |
| pcDNA3.1(+)-E | BamHI/EcoRI cut E fragment(~300 bp), from | BamHI/EcoRI cut |
| | pMD18-T/E | pcDNA3.1(+) |
| pcDNA3.1(+)-M | BamHI/EcoRI cut M fragment(~760 bp), | BamHI/EcoRI cut |
| | pMD18-T/M | pcDNA3.1(+) |
| pcDNA3.1(+)-N | BamHI/EcoRI cut N fragement(~1315 bp), | BamHI/EcoRI cut |
| | pMD18-T/N | pcDNA3.1(+) |
| pcDNA3.1(−)-X2 | KpnI/EcoRI cut X2 fragement(~380 bp) | KpnI/EcoRI cut pcDNA3.1 |
| | pMD18-T/X2 | (−) |

### Example 3

### Immunological Tests of the Nucleotide Sequence Coding SARS Coronavirus (CoV-SARS) E, M, S, X and N Protein on Mice

To control the occurrence and spreading of SARS, it is very important to research on the virus vaccine. Comparing to regular attenuated and atrophic vaccines, DNA vaccine is much more preferred. The new vaccine had no immunogen, very effective, long-lasting, easy producing and using, easy storing, and low production cost. It had not been reported that DNA vaccine plasmids compliced into host animal genomes.

The complex adenovirus vector system was used in immunogen testings for the nucleotide sequence which codes small envelope membrane protein (E), small membrane protein (M), spike protein or glycoprotein, and nuclear capsid protein (N). All of the adenovirus vectors contained E3, missed E4 ORF6, except for ORF6.

The DNA vaccine of the invention was produced using complex adenovirus vector system as carrier. The S1 vaccine was made by inserting into the vector nucleotide fragment for coding SARS virus S protein. The S2 vaccine was made by inserting into the vector nucleotides fragment for coding SARS virus S and E proteins. The S3N vaccine was made by inserting into the vector nucleotide fragments for coding SARS virus E, M and N proteins. The S3G vaccine was made by inserting into the vector nucleotides fragment for coding SARS virus S, E and M proteins.

The vaccines were tested on mice. Each group (6) of mice was injected with all candidate vaccines with volume of 10^{8th} pfu for each. The blood samples were obtained once every two weeks. Antibodies for S, E proteins were tested using ELISA.

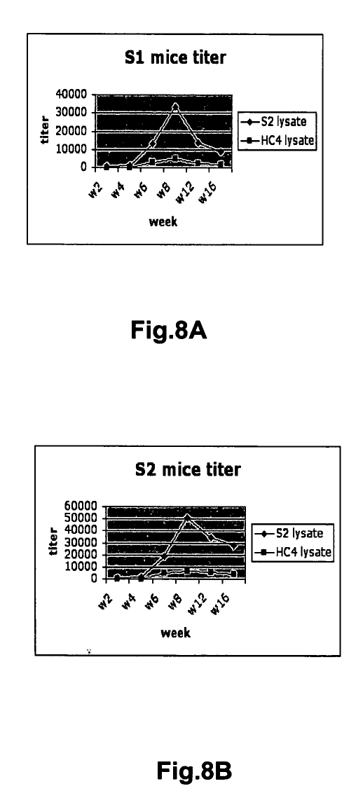

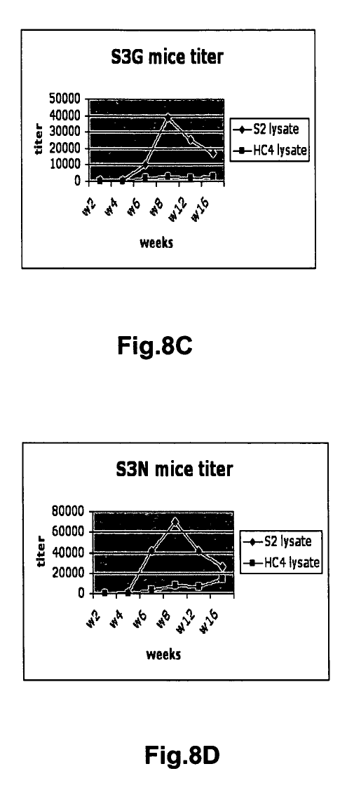

The experiment results were shown in FIG. 8A-8D. S1 was the complex adenovirus vector which only expressed spike protein (see FIG. 8A); S2 was the vector which expressed S and E proteins; S3G was the vector which expressed S, M and E proteins (see FIG. 8C); S3N was the vector which expressed E, M and N proteins (see FIG. 8D). The S2 lysis was the broken cells which expressed S and E proteins. The cells were human A549 lung cancer cells which were transfected with the vectors comprising S and E proteins. The cell matrix were used as immunological target and used to coat the wells on ELISA plate. There were some irrelevant antibodies in HC4 lysis, which was used as control.

### Example 4

### Repetitive Experiments of S3N Vaccine in Mice

The preparation methods were used to make S3N vaccine. The vaccine was used in this repetitive experiment and injected into mice intraperitoneally.

Materials

1. The animals were C57 mice from Shanghai Silaike Lab Animal Ltd Corp. The mice were kept in lab animal room with regular day/night rotation. The mice were half male and half female. All were about 8 weeks. The body weight was between 19 g to 29 g when the blood sample was first obtained.

2. SARS IgG antibody ELISA kit was purchased from Beijing Huadajiebiai Biotech Ltd Corp.

Methods

1. Procedures of the Animal Experiments:
1) Administration: C57 mice of 8 weeks were divided up to two groups with 10 in each group. Mice of each group were injected intraperitoneally with vaccine S3N. The control group was injected with PBS. The vaccine S3N was dissolved in PBS with concentration of 10⁸ pfu/ml. Each mouse was injected with 0.5 ml of the vaccine.
2) Sera preparation: 100 μl blood samples were obtained from orbit of the mice at 0 week (before administration), 2 weeks, 4 weeks, 6 weeks, 8 weeks (before administration), 10 weeks, 12 weeks, and 16 weeks. The blood samples were sat in room temperature for 1 hour. The sera were obtained after the blood samples being centrifuged and stored at −20° C.
3) Testing SARS antibody in sera samples: The ELISA used in this experiment for testing anti SARS antibody, was a modified method based on ELISA kit from Beijing Huadajibiai Biotech Ltd Corp. The references included Himani Bisht, Anjeanette Roberts, et al. Severe acute respiratory syndrome coronavirus spike protein expressed by attenuated vaccinia virus protectively immunizes mice. PNAS Apr. 27, 2004, vol. 101 no. 17 6641-6646; and Wenlin Huang, Ranyi Liu, Bijun Huang, and Jialin Huang, Construction of Recombinant Adenovirus of Spike Gene Fragment And Its Immunological Reaction for Anti SARS-CoV.

2. Procedures:
a). Every experimental well of the kit were incubated with 5% BSA dissolved in PBS (PH 7.5) at 37° C. for 60 minutes.
b). The experiment plate was washed 5 times with the buffer provided in the kit. Diluted sera samples were then added on the plate. The samples were diluted in different concentrations starting at 1/50. The positive and negative controls were also added. And the plate was incubated in 37° C. for 60 minutes.
c). The experiment plate was washed 5 times with the buffer provided in the kit. The following mixtures were added into each well of the plate: 0.5 μg/ml goat anti mice IgG-HRP+0.2% tween 20+1% BSA diluted in PBS. The plate was then incubated in 37° C. for minutes.
d). The experiment plate was washed 5 times with the buffer provided in the kit and 0.05% tween 20. The development was applied according to the description of the kit. The time of the development was controlled in 5˜10 minutes.
e). Double-wavelength Testing: 450 nm, 630 nm.
The dates of DNA SARS vaccine injections and the blood sample

| Group | S3N | PBS | |
|---|---|---|---|
| Sera preparation before injection 1 | 01/16 | 01/16 | |
| Sera preparation before injection 2 | 02/18 | 02/18 | |
| First injection (week 0) | 02/19 | 02/19 | |
| First blood obtaining (week 2) | 03/03 | 03/03 | |
| Second blood obtaining (week 4) | 03/17 | 03/17 | ELISA 3 times sera |
| | | | sample |
| Third blood obtaining (week 6) | 03/31 | 03/31 | |
| Fourth blood obtaining (week 8) | 04/14 | 04/14 | ELISA |
| preinjection | | | |
| Second injection (week 8) | 04/14 | 04/14 | |
| Fifth blood obtaining (week 10) | 04/28 | 04/28 | |
| Sixth blood obtaining (week 12) | 05/12 | 05/12 | |
| Seventh blood obtaining (week 14) | 05/27 | 05/27 | ELISA 8 times sera |
| half of the mice were killed | | | sample |

obtaining were listed in the table below:

Results:

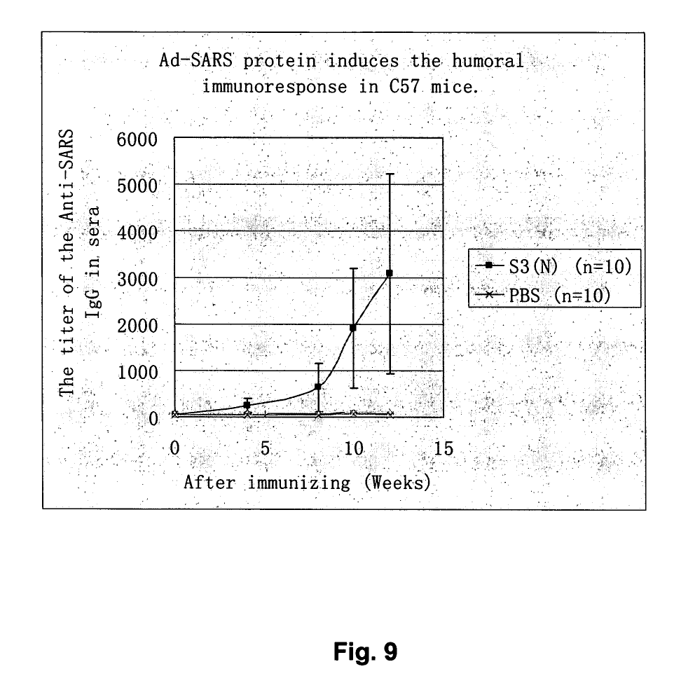

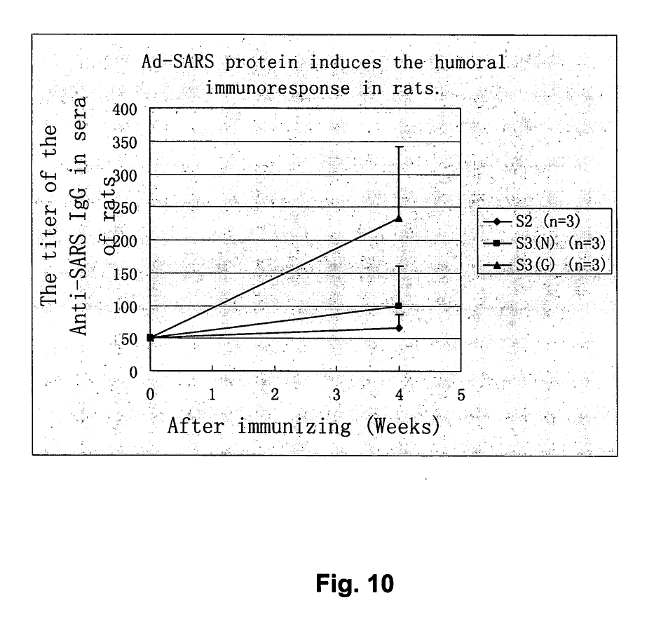

The anti SARS IgG titers in sera of mice at 0, 4, 8, 10 and 12 weeks, were tested using ELISA. All samples were diluted. The results shown in the FIG. 9 included: 1. immunological reaction in mice body fluid was induced 4 weeks after Ad-S3N injection; 2. immunological reaction in mice body fluid was strengthened at 8th week after Ad-S3N reinjection, with high titer of 3000.

### Example 5

### Testing the Immunological Effect of SARS DNA Vaccine S2, S3N and S3G for Rats

The adenovirus in this experiment was the vector for the DNA vaccines. S2 vaccine was made by inserting into the vector with gene fragment coding SARS virus protein S and E. S3N vaccine was made by inserting into the vector with gene fragment coding SARS virus protein E, M and N. S3G vaccine was made by inserting into the vector with gene fragment coding SARS virus protein S, E and M. There were different methods for DNA vaccines. Intraperitoneal injection was used in this experiment.

Object:

To test the immunological induction of SARS DNA vaccine S2, S3(N), S3(G) in rats.

Materials:
1. The lab animals used in this experiment were purchased from Shanghai Silaike Lab Animal Ltd Corp. The animals were kept in animal room of the lab, with regular day/night alternates. Rats were all males with body weight of 200 g.
2. SARS IgG antibody ELISA kit was purchased from Beijing Huadajiebiai Biotech Ltd Corp.

Methods:

1. Animal Experiment Procedures:
1) Administration: SD rats, male, body weight of 2009 were divided into 4 groups. 3 of the groups were administration group with 3 rats per group, while 1 of the groups was the PBS injection control group with only 1 rat. Each group was injected intraperitoneally with vaccine S2, S3(G), S3(G) and PBS control. All vaccines were dissolved in PBS. The volume of injection was 109 pfu per rat.
2) Sera preparation: 200 μl blood samples were obtained from tails at 0 week (preinjection), 4 weeks, 8 weeks (preinjection), 10 weeks, 12 weeks, and 16 weeks. Samples were in room temperature for 1 hour. The sera was obtained after centrifugation and stored at −20° C.
3) Testing SARS antibody in sera: the SARS antibody ELISA test was a modified method based on ELISA kit purchased from Beijing Huadajiebiai Biotech Ltd Corp. Referred to the description before.

2. Procedures:
a). Every experimental well of the kit was incubated together with 5% BSA dissolved in PBS (PH7.5) in 37° C. for 60 minutes.
b). The experiment plate was washed 5 times with the buffer provided in the kit. Diluted sera samples were then added on the plate. The samples were diluted in different concentrations starting at 1/50. The positive and negative controls were also added. And the plate was incubated in 37° C. for 60 minutes.
c). The experiment plate was washed 5 times with the buffer provided in the kit. The following mixtures were added into each well of the plate: 0.5 μg/ml lamb anti mice IgG-HRP+0.2% tween 20+1% BSA diluted in PBS. The plate was then incubated in 37° C. for 60 minutes.
d). The experiment plate was washed 5 times with the buffer provided in the kit and 0.05% tween 20. The development was applied according to the description of the kit. The time of the development was controlled in 5˜10 minutes.
e). Double-wavelength Testing: 450 nm, 630 nm.
The dates of the injection of DNA SARS vaccine injections and the blood sample obtaining were listed in the table below:

| Group | S2 | S3N | S3G | PBS |
|---|---|---|---|---|
| Sera preparation before injection 1 | 04/08 | 04/08 | 04/08 | 04/08 |
| First injection (week 0) | 04/08 | 04/08 | 04/08 | 04/08 |
| First blood obtaining (week 4) | 05/09 | 05/09 | 05/09 | 05/09 |
| Sencond blood obtaining + injection | 06/03 | 06/03 | 06/03 | 06/03 |
| (week 8) | | | | |
| Third blodd obtaining (week 10) | 06/17 | 06/17 | 06/17 | 06/17 |
| Fourth blood obtaining (week 12) | 07/01 | 07/01 | 07/01 | 07/01 |

Results:

The anti SARS IgG titers in sera of rats at 0 and 4 weeks, were tested using ELISA. All samples were diluted. The results shown in the FIG. 10 included: rat body fluid immunological reactions could be induced by Ad-S3G, and its titer at 4^{th} week could be more than 200.

1-93. (canceled)

94. An isolated polynucleotide selected from the group consisting of:
a. a polynucleotide sequence of SEQ ID NO: 1;
b. a naturally-occurring polynucleotide sequence having at least 90% sequence identity to the sequence of SEQ ID NO: 1; and
c. a polynucleotide sequence complementary to either a) or b).

95. An isolated polynucleotide selected from the group consisting of:
a. a polynucleotide sequence selected from the group consisting of SEQ ID NOs: 2-7;
b. a naturally-occurring polynucleotide sequence having at least 90% sequence identity to a sequence selected from the group consisting of SEQ ID NOs: 2-7; and
c. a polynucleotide sequence complementary to either a) or b).

96. An isolated polypeptide sequence comprising an amino acid sequence selected from the group consisting of:
a. an amino acid sequence as set forth in any of SEQ ID NOS. 8, 28-37;
b. a naturally-occurring amino acid sequence having at least 90% sequence identity to any of the amino acid sequence of SEQ ID NOS. 8, 28-37;
c. a biologically active fragment of any of the amino acid sequence of SEQ ID NOS. 8, 28-37; and
d. an immunogenic fragment of the amino acid sequence of SEQ ID NOS. 8, 28-37.

97. An isolated polypeptide fragment capable of generating an immune response against the SARS virus selected from the group consisting of
a. a polypeptide sequence selected from the group consisting of SEQ ID NOs: 9-14;
b. a naturally-occurring polypeptide sequence having at least 90% sequence identity to a sequence selected from the group consisting of SEQ ID NOs: 9-14.

98. A vaccine effective against a human SARS virus infection comprising a peptide having a sequence selected from the group consisting of SEQ ID NOs: 1-7 and a pharmaceutically acceptable carrier.

99. A vaccine effective against a human SARS virus infection comprising a peptide having a sequence selected from the group consisting of SEQ ID NOs: 8-14, 28-37 and a pharmaceutically acceptable carrier.

100. A recombinant adenovirus expressing SARS viral proteins, comprising:
a. an adenovirus, wherein portions of its sequence responsible for replication having been deleted, thus rending the adenovirus incapable of replicating itself; and
b. at least one polypeptide fragment selected from the group consisting of the spike protein, the small membrane protein, the small envelope protein, and the nuclear capsid protein.

101. A recombinant adenovirus expressing SARS viral proteins, comprising:
a. an adenovirus, wherein portions of its sequence responsible for replication having been deleted, thus rending the adenovirus incapable of replicating itself; and
b. two polypeptide fragments selected from the group consisting of the spike protein, the small membrane protein, the small envelope protein, and the nuclear capsid protein.

102. A recombinant adenovirus expressing SARS viral proteins, comprising:
a. an adenovirus, wherein portions of its sequence responsible for replication having been deleted, thus rending the adenovirus incapable of replicating itself; and
b. three polypeptide fragments selected from the group consisting of the spike protein, the small membrane protein, the small envelope protein, and the nuclear capsid protein.

103. A recombinant adenovirus expressing SARS viral proteins, comprising:
a. an adenovirus, wherein portions of its sequence responsible for replication having been deleted, thus rending the adenovirus incapable of replicating itself; and
b. a plurality of polypeptide fragments selected from the group consisting of the spike protein, the small membrane protein, the small envelop protein, and the nuclear capsid protein.

104. A SARS vaccine comprising of the recombinant adenovirus of claim 100, and a pharmaceutically acceptable carrier.

105. A method of modulating the immune response to human SARS virus infection, comprising administering an effective amount of the vaccine according to claim 104.

106. A method of immunizing a subject against a SARS virus infection comprising administering to said subject the vaccine of claim 104.

107. The method of claim 106, wherein said subject is a human.


SEQUENCE LISTING (37 sequences)

SEQ ID NO: 1 - DNA, 29760 bp, Sars coronaviruses (SARS-CoV)
atattaggtttttacctacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcactgccacaaggtcgttgagctggttgcagaaatggacggcattcagtacggtcgtagcggtataacactgggagtactcgtgccacatgtgggcgaaaccccaattgcataccgcaatgttcttcttcgtaagaacggtaataagggagccggtggtcatagctatggcatcgatctaaagtcttatgacttaggtgacgagcttggcactgatcccattgaagattatgaacaaaactggaacactaagcatggcagtggtgcactccgtgaactcactcgtgagctcaatggaggtgcagtcactcgctatgtcgacaacaatttctgtggcccagatgggtaccctcttgattgcatcaaagattttctcgcacgcgcgggcaagtcaatgtgcactctttccgaacaacttgattacatcgagtcgaagagaggtgtctactgctgccgtgaccatgagcatgaaattgcctggttcactgagcgctctgataagagctacgagcaccagacacccttcgaaattaagagtgccaagaaatttgacactttcaaaggggaatgcccaaagtttgtgtttcctcttaactcaaaagtcaaagtcattcaaccacgtgttgaaaagaaaaagactgagggtttcatggggcgtatacgctctgtgtaccctgttgcatctccacaggagtgtaacaacatgcacttgtctaccttgatgaaatgtaatcattgcgatgaagtttcatggcagacgtgcgactttctgaaagccacttgtgaacattgtggcactgaaaatttagttattgaaggacctactacatgtgggtacctacctactaatgctgtagtgaaaatgccatgtcctgcctgtcaagacccagagattggacctgagcatagtgttgcagattatcacaaccactcaaacattgaaactcgactccgcaagggaggtaggactagatgttttggaggctgtgtgtttgcctatgttggctgctataataagcgtgcctactgggttcctcgtgctagtgctgatattggctcaggccatactggcattactggtgacaatgtggagaccttgaatgaggatctccttgagatactgagtcgtgaacgtgttaacattaacattgttggcgattttcatttgaatgaagaggttgccatcattttggcatctttctctgcttctacaagtgcctttattgacactataaagagtcttgattacaagtctttcaaaaccattgttgagtcctgcggtaactataaagttaccaagggaaagcccgtaaaaggtgcttggaacattggacaacagagatcagttttaacaccactgtgtggttttccctcacaggctgctggtgttatcagatcaatttttgcgcgcacacttgatgcagcaaaccactcaattcctgatttgcaaagagcagctgtcaccatacttgatggtatttctgaacagtcattacgtcttgtcgacgccatggtttatacttcagacctgctcaccaacagtgtcattattatggcatatgtaactggtggtcttgtacaacagacttctcagtggttgtctaatcttttgggcactactgttgaaaaactcaggcctatctttgaatggattgaggcgaaacttagtgcaggagttgaatttctcaaggatgcttgggagattctcaaatttctcattacaggtgtttttgacatcgtcaagggtcaaatacaggttgcttcagataacatcaaggattgtgtaaaatgcttcattgatgttgttaacaaggcactcgaaatgtgcattgatcaagtcactatcgctggcgcaaagttgcgatcactcaacttaggtgaagtcttcatcgctcaaagcaagggactttaccgtcagtgtatacgtggcaaggagcagctgcaactactcatgcctcttaaggcaccaaaagaagtaacctttcttgaaggtgattcacatgacacagtacttacctctgaggaggttgttctcaagaacggtgaactcgaagcactcgagacgcccgttgatagcttcacaaatggagctatcgttggcacaccagtctgtgtaaatggcctcatgctcttagagattaaggacaaagaacaatactgcgcattgtctcctggtttactggctacaaacaatgtctttcgcttaaaagggggtgcaccaattaaaggtgtaacctttggagaagatactgtttgggaagttcaaggttacaagaatgtgagaatcacatttgagcttgatgaacgtgttgacaaagtgcttaatgaaaagtgctctgtctacactgttgaatccggtaccgaagttactgagtttgcatgtgttgtagcagaggctgttgtgaagactttacaaccagtttctgatctccttaccaacatgggtattgatcttgatgagtggagtgtagctacattctacttatttgatgatgctggtgaagaaaacttttcatcacgtatgtattgttccttttaccctccagatgaggaagaagaggacgatgcagagtgtgaggaagaagaaattgatgaaacctgtgaacatgagtacggtacagaggatgattatcaaggtctccctctggaatttggtgcctcagctgaaacagttcgagttgaggaagaagaagaggaagactggctggatgatactactgagcaatcagagattgagccagaaccagaacctacacctgaagaaccagttaatcagtttactggttatttaaaacttactgacaatgttgccattaaatgtgctgacatcgttaaggaggcacaaagtgctaatcctatggtgattgtaaatgctgctaacatacacctgaaacatggtggtggtgtagcaggtgcactcaacaaggcaaccaatggtgccatgcaaaaggagagtgatgattacattaagctaaatggccctcttacagtaggagggtcttgtttgctttctggacataatcttgctaagaagtgtctgcatgttgttggacctaacctaaatgcaggtgaggacatccagcttcttaaggcagcatatgaaaatttcaattcacaggacaccttacttgcaccattgttgtcagcaggcatatttggtgctaaaccacttcagtctttacaagtgtgcgtgcagacggttcgtacacaggtttatattgcagtcaatgacaaagctctttatgagcaggttgtcatggattatcttgataacctgaagcctagagtggaagcacctaaacaagaggagccaccaaacacagaagattccaaaactgaggagaaatctgtcgtacagaagcctgtcgatgtgaagccaaaaattaaggcctgcattgatgaggttaccacaacactggaagaaactaagtttcttaccaataagttactcttgtttgctgatatcaatggtaagctttaccatgattctcagaacatgcttagaggtgaagatatgtctttccttgagaaggatgcaccttacatggtaggtgatgttatcactagtggtgatatcacttgtgttgtaataccctccaaaaaggctggtggcactactgagatgctctcaagagctttgaagaaagtgccagttgatgagtatataaccacgtaccctggacaaggatgtgctggttatacacttgaggaagctaggactgctcttaagaaatgcaaatctgcattttatgtactaccttcagaagcacctaatgctaaggaagagattctaggaactgtatcctggaatttgagagaaatgcttgctcatgctgaagagacaagaaaattaatgcctatatgcatggatgttagagccataatggcaaccatccaacgtaagtataaaggaattaaaattcaagagggcatcgttgactatggtgtccgattcttcttttatactagtaaagagcctgtagcttctattattacgaagctgaactctctaaatgagccgcttgtcacaatgccaattggttatgtgacacatggttttaatcttgaagaggctgcgcgctgtatgcgttctcttaaagctcctgccgtagtgtcagtatcatcaccagatgctgttactacatataatggatacctcacttcgtcatcaaagacatctgaggagcactttgtagaaacagtttctttggctggctcttacagagattggtcctattcaggacagcgtacagagttaggtgttgaatttcttaagcgtggtgacaaaattgtgtaccacactctggagagccccgtcgagtttcatcttgacggtgaggttctttcacttgacaaactaaagagtctcttatccctgcgggaggttaagactataaaagtgttcacaactgtggacaacactaatctccacacacagcttgtggatatgtctatgacatatggacagcagtttggtccaacatacttggatggtgctgatgttacaaaaattaaacctcatgtaaatcatgagggtaagactttctttgtactacctagtgatgacacactacgtagtgaagctttcgagtactaccatactcttgatgagagttttcttggtaggtacatgtctgctttaaaccacacaaagaaatggaaatttcctcaagttggtggtttaacttcaattaaatgggctgataacaattgttatttgtctagtgttttattagcacttcaacagattgaagtcaaattcaatgcaccagcacttcaagaggcttattatagagcccgtgctggtgatgctgctaacttttgtgcactcatactcgcttacagtaataaaactgttggcgagcttggtgatgtcagagaaactatgacccatcttctacagcatgctaatttggaatctgcaaagcgagttcttaatgtggtgtgtaaacattgtggtcagaaaactactaccttaacgggtgtagaagctgtgatgtatatgggtactctatcttatgataatcttaagacaggtgtttccattccatgtgtgtgtggtcgtgatgctacacaatatctagtacaacaagagtcttcttttgttatgatgtctgcaccacctgctgagtataaattacagcaaggtacattcttatgtgcgaatgagtacactggtaactatcagtgtggtcattacactcatataactgctaaggagaccctctatcgtattgacggagctcaccttacaaagatgtcagagtacaaaggaccagtgactgatgttttctacaaggaaacatcttacactacaaccatcaagcctgtgtcgtataaactcgatggagttacttacacagagattgaaccaaaattggatgggtattataaaaaggataatgcttactatacagagcagcctatagaccttgtaccaactcaaccattaccaaatgcgagttttgataatttcaaactcacatgttctaacacaaaatttgctgatgatttaaatcaaatgacaggcttcacaaagccagcttcacgagagctatctgtcacattcttcccagacttgaatggcgatgtagtggctattgactatagacactattcagcgagtttcaagaaaggtgctaaattactgcataagccaattgtttggcacattaaccaggctacaaccaagacaacgttcaaaccaaacacttggtgtttacgttgtctttggagtacaaagccagtagatacttcaaattcatttgaagttctggcagtagaagacacacaaggaatggacaatcttgcttgtgaaagtcaacaacccacctctgaagaagtagtggaaaatcctaccatacagaaggaagtcatagagtgtgacgtgaaaactaccgaagttgtaggcaatgtcatacttaaaccatcagatgaaggtgttaaagtaacacaagagttaggtcatgaggatcttatggctgcttatgtggaaaacacaagcattaccattaagaaacctaatgagctttcactagccttaggtttaaaaacaattgccactcatggtattgctgcaattaatagtgttccttggagtaaaatttttgcttatgtcaaaccattcttaggacaagcagcaattacaacatcaaattgcgctaagagattagcacaacgtgtgtttaacaattatatgccttatgtgtttacattattgttccaattgtgtacttttactaaaagtaccaattctagaattagagcttcactacctacaactattgctaaaaatagtgttaagagtgttgctaaattatgtttggatgccggcattaattatgtgaagtcacccaaattttctaaattgttcacaatcgctatgtggctattgttgttaagtatttgcttaggttctctaatctatgtaactgctgcttttggtgtactcttatctaattttggtgctccttcttattgtaatggcgttagagaattgtatcttaattcgtctaacgttactactatggatttctgtgaaggttcttttccttgcagcatttgtttaagtggattagactcccttgattcttatccagctcttgaaaccattcaggtgacgatttcatcgtacaagctagacttgacaattttaggtctggccgctgagtgggttttggcatatatgttgttcacaaaattcttttatttattaggtctttcagctataatgcaggtgttctttggctattttgctagtcatttcatcagcaattcttggctcatgtggtttatcattagtattgtacaaatggcacccgtttctgcaatggttaggatgtacatcttctttgcttctttctactacatatggaagagctatgttcatatcatggatggttgcacctcttcgacttgcatgatgtgctataagcgcaatcgtgccacacgcgttgagtgtacaactattgttaatggcatgaagagatctttctatgtctatgcaaatggaggccgtggcttctgcaagactcacaattggaattgtctcaattgtgacacattttgcactggtagtacattcattagtgatgaagttgctcgtgatttgtcactccagtttaaaagaccaatcaaccctactgaccagtcatcgtatattgttgatagtgttgctgtgaaaaatggcgcgcttcacctctactttgacaaggctggtcaaaagacctatgagagacatccgctctcccattttgtcaatttagacaatttgagagctaacaacactaaaggttcactgcctattaatgtcatagtttttgatggcaagtccaaatgcgacgagtctgcttctaagtctgcttctgtgtactacagtcagctgatgtgccaacctattctgttgcttgaccaagctcttgtatcagacgttggagatagtactgaagtttccgttaagatgtttgatgcttatgtcgacaccttttcagcaacttttagtgttcctatggaaaaacttaaggcacttgttgctacagctcacagcgagttagcaaagggtgtagctttagatggtgtcctttctacattcgtgtcagctgcccgacaaggtgttgttgataccgatgttgacacaaaggatgttattgaatgtctcaaactttcacatcactctgacttagaagtgacaggtgacagttgtaacaatttcatgctcacctataataaggttgaaaacatgacgcccagagatcttggcgcatgtattgactgtaatgcaaggcatatcaatgcccaagtagcaaaaagtcacaatgtttcactcatctggaatgtaaaagactacatgtctttatctgaacagctgcgtaaacaaattcgtagtgctgccaagaagaacaacataccttttagactaacttgtgctacaactagacaggttgtcaatgtcataactactaaaatctcactcaagggtggtaagattgttagtacttggtttaaacttatgcttaaggccacattattgtgcgttcttgctgcattggtttgttacatcgttatgccagtacatacattgtcaatccatgatggttacacaaatgaaatcattggttacaaagccattcaggatggtgtcactcgtgacatcatttctactgatgattgttttgcaaataaacatgctggttttgacgcatggtttagccagcgtggtggttcatacaaaaatgacaaaagctgccctgtagtagctgctatcattacaagagagattggtttcatagtgcctggcttaccgggtactgtgttgagagcaatcaatggtgacttcttgcattttctacctcgtgtttttagtgctgttggcaacatttgctacacaccttccaaactcattgagtatagtgattttgctacctctgcttgcgttcttgctgcagagtgtacaatttttaaggatgctatgggcaaacctgtgccatattgttatgacactaatttgctagagggttctatttcttatagtgagcttcgtccagacactcgttatgtgcttatggatggttccatcatacagtttcctaacatttacctggagggttctgttagagtagtaacaacttttgatgctgagtactgtagacatggtacatgcgaaaggtcagaagcaggtatttgcctatctaccagtggtagatgggttcttaataatgagcattacagagctctatcaggagttttctgtggtgttgatgcgatgaatctcatagctaacatctttactcctcttgtgcaacctgtgggtgctttagatgtgtctgcttcagtagtggctggtggtattattgccatattggtgacttgtgctgcctactactttatgaaattcagacgtgcttttggtgagtacaaccatgttgttgctgctaatgcacttttgtttttgatgtctttcactatactctgtctggcaccagcttacagctttctgccgggagtctactcagtcttttacttgtacttgacattctatttcaccaatgatgtttcattcttggctcaccttcaatggtttgccatgttttctcctattgtgcctttttggataacagcaatctatgtattctgtatttctctgaagcactgccattggttctttaacaactatcttaggaaaagagtcatgtttaatggagttacatttagtaccttcgaggaggctgctttgtgtacctttttgctcaacaaggaaatgtacctaaaattgcgtagcgagacactgttgccacttacacagtataacaggtatcttgctctatataacaagtacaagtatttcagtggagccttagatactaccagctatcgtgaagcagcttgctgccacttagcaaaggctctaaatgactttagcaactcaggtgctgatgttctctaccaaccaccacagacatcaatcacttctgctgttctgcagagtggttttaggaaaatggcattcccgtcaggcaaagttgaaggatgcatggtacaagtaacctgtggaactacaactcttaatggattgtggttggatgacacagtatactgtccaagacatgtcatttgcacagcagaagacatgcttaatcctaactatgaagatctgctcattcgcaaatccaaccatagctttcttgttcaggctggcaatgttcaacttcgtgttattggccattctatgcaaaattgtctgcttaggcttaaagttgatacttctaaccctaagacacccaagtataaatttgtccgtatccaacctggtcaaacattttcagttctagcatgctacaatggttcaccatctggtgtttatcagtgtgccatgagacctaatcataccattaaaggttctttccttaatggatcatgtggtagtgttggttttaacattgattatgattgcgtgtctttctgctatatgcatcatatggagcttccaacaggagtacacgctggtactgacttagaaggtaaattctatggtccatttgttgacagacaaactgcacaggctgcaggtacagacacaaccataacattaaatgttttggcatggctgtatgctgctgttatcaatggtgataggtggtttcttaatagattcaccactactttgaatgactttaaccttgtggcaatgaagtacaactatgaacctttgacacaagatcatgttgacatattgggacctctttctgctcaaacaggaattgccgtcttagatatgtgtgctgctttgaaagagctgctgcagaatggtatgaatggtcgtactatccttggtagcactattttagaagatgagtttacaccatttgatgttgttagacaatgctctggtgttaccttccaaggtaagttcaagaaaattgttaagggcactcatcattggatgcttttaactttcttgacatcactattgattcttgttcaaagtacacagtggtcactgtttttctttgtttacgagaatgctttcttgccatttactcttggtattatggcaattgctgcatgtgctatgctgcttgttaagcataagcacgcattcttgtgcttgtttctgttaccttctcttgcaacagttgcttactttaatatggtctacatgcctgctagctgggtgatgcgtatcatgacatggcttgaattggctgacactagcttgtctggttataggcttaaggattgtgttatgtatgcttcagctttagttttgcttattctcatgacagctcgcactgtttatgatgatgctgctagacgtgtttggacactgatgaatgtcattacacttgtttacaaagtctactatggtaatgctttagatcaagctatttccatgtgggccttagttatttctgtaacctctaactattctggtgtcgttacgactatcatgtttttagctagagctatagtgtttgtgtgtgttgagtattacccattgttatttattactggcaacaccttacagtgtatcatgcttgtttattgtttcttaggctattgttgctgctgctactttggccttttctgtttactcaaccgttacttcaggcttactcttggtgtttatgactacttggtctctacacaagaatttaggtatatgaactcccaggggcttttgcctcctaagagtagtattgatgctttcaagcttaacattaagttgttgggtattggaggtaaaccatgtatcaaggttgctactgtacagtctaaaatgtctgacgtaaagtgcacatctgtggtactgctctcggttcttcaacaacttagagtagagtcatcttctaaattgtgggcacaatgtgtacaactccacaatgatattcttcttgcaaaagacacaactgaagctttcgagaagatggtttctcttttgtctgttttgctatccatgcagggtgctgtagacattaataggttgtgcgaggaaatgctcgataaccgtgctactcttcaggctattgcttcagaatttagttctttaccatcatatgccgcttatgccactgcccaggaggcctatgagcaggctgtagctaatggtgattctgaagtcgttctcaaaaagttaaagaaatctttgaatgtggctaaatctgagtttgaccgtgatgctgccatgcaacgcaagttggaaaagatggcagatcaggctatgacccaaatgtacaaacaggcaagatctgaggacaagagggcaaaagtaactagtgctatgcaaacaatgctcttcactatgcttaggaagcttgataatgatgcacttaacaacattatcaacaatgcgcgtgatggttgtgttccactcaacatcataccattgactacagcagccaaactcatggttgttgtccctgattatggtacctacaagaacacttgtgatggtaacacctttacatatgcatctgcactctgggaaatccagcaagttgttgatgcggatagcaagattgttcaacttagtgaaattaacatggacaattcaccaaatttggcttggcctcttattgttacagctctaagagccaactcagctgttaaactacagaataatgaactgagtccagtagcactacgacagatgtcctgtgcggctggtaccacacaaacagcttgtactgatgacaatgcacttgcctactataacaattcgaagggaggtaggtttgtgctggcattactatcagaccaccaagatctcaaatgggctagattccctaagagtgatggtacaggtacaatttacacagaactggaaccaccttgtaggtttgttacagacacaccaaaagggcctaaagtgaaatacttgtacttcatcaaaggcttaaacaacctaaatagaggtatggtgctgggcagtttagctgctacagtacgtcttcaggctggaaatgctacagaagtacctgccaattcaactgtgctttccttctgtgcttttgcagtagaccctgctaaagcatataaggattacctagcaagtggaggacaaccaatcaccaactgtgtgaagatgttgtgtacacacactggtacaggacaggcaattactgtaacaccagaagctaacatggaccaagagtcctttggtggtgcttcatgttgtctgtattgtagatgccacattgaccatccaaatcctaaaggattctgtgacttgaaaggtaagtacgtccaaatacctaccacttgtgctaatgacccagtgggttttacacttagaaacacagtctgtaccgtctgcggaatgtggaaaggttatggctgtagttgtgaccaactccgcgaacccttgatgcagtctgcggatgcatcaacgtttttaaacgggtttgcggtgtaagtgcagcccgtcttacaccgtgcggcacaggcactagtactgatgtcgtctacagggcttttgatatttacaacgaaaaagttgctggttttgcaaagttcctaaaaactaattgctgtcgcttccaggagaaggatgaggaaggcaatttattagactcttactttgtagttaagaggcatactatgtctaactaccaacatgaagagactatttataacttggttaaagattgtccagcggttgctgtccatgactttttcaagtttagagtagatggtgacatggtaccacatatatcacgtcagcgtctaactaaatacacaatggctgatttagtctatgctctacgtcattttgatgagggtaattgtgatacattaaaagaaatactcgtcacatacaattgctgtgatgatgattatttcaataagaaggattggtatgacttcgtagagaatcctgacatcttacgcgtatatgctaacttaggtgagcgtgtacgccaatcattattaaagactgtacaattctgcgatgctatgcgtgatgcaggcattgtaggcgtactgacattagataatcaggatcttaatgggaactggtacgatttcggtgatttcgtacaagtagcaccaggctgcggagttcctattgtggattcatattactcattgctgatgcccatcctcactttgactagggcattggctgctgagtcccatatggatgctgatctcgcaaaaccacttattaagtgggatttgctgaaatatgattttacggaagagagactttgtctcttcgaccgttattttaaatattgggaccagacataccatcccaattgtattaactgtttggatgataggtgtatccttcattgtgcaaactttaatgtgttattttctactgtgtttccacctacaagttttggaccactagtaagaaaaatatttgtagatggtgttccttttgttgtttcaactggataccattttcgtgagttaggagtcgtacataatcaggatgtaaacttacatagctcgcgtctcagtttcaaggaacttttagtgtatgctgctgatccagctatgcatgcagcttctggcaatttattgctagataaacgcactacatgcttttcagtagctgcactaacaaacaatgttgcttttcaaactgtcaaacccggtaattttaataaagacttttatgactttgctgtgtctaaaggtttctttaaggaaggaagttctgttgaactaaaacacttcttctttgctcaggatggcaacgctgctatcagtgattatgactattatcgttataatctgccaacaatgtgtgatatcagacaactcctattcgtagttgaagttgttgataaatactttgattgttacgatggtggctgtattaatgccaaccaagtaatcgttaacaatctggataaatcagctggtttcccatttaataaatggggtaaggctagactttattatgactcaatgagttatgaggatcaagatgcacttttcgcgtatactaagcgtaatgtcatccctactataactcaaatgaatcttaagtatgccattagtgcaaagaatagagctcgcaccgtagctggtgtctctatctgtagtactatgacaaatagacagtttcatcagaaattattgaagtcaatagccgccactagaggagctactgtggtaattggaacaagcaagttttacggtggctggcataatatgttaaaaactgtttacagtgatgtagaaactccacaccttatgggttgggattatccaaaatgtgacagagccatgcctaacatgcttaggataatggcctctcttgttcttgctcgcaaacataacacttgctgtaacttatcacaccgtttctacaggttagctaacgagtgtgcgcaagtattaagtgagatggtcatgtgtggcggctcactatatgttaaaccaggtggaacatcatccggtgatgctacaactgcttatgctaatagtgtctttaacatttgtcaagctgttacagccaatgtaaatgcacttctttcaactgatggtaataagatagctgacaagtatgtccgcaatctacaacacaggctctatgagtgtctctatagaaatagggatgttgatcatgaattcgtggatgagttttacgcttacctgcgtaaacatttctccatgatgattctttctgatgatgccgttgtgtgctataacagtaactatgcggctcaaggtttagtagctagcattaagaactttaaggcagttctttattatcaaaataatgtgttcatgtctgaggcaaaatgttggactgagactgaccttactaaaggacctcacgaattttgctcacagcatacaatgctagttaaacaaggagatgattacgtgtacctgccttacccagatccatcaagaatattaggcgcaggctgttttgtcgatgatattgtcaaaacagatggtacacttatgattgaaaggttcgtgtcactggctattgatgcttacccacttacaaaacatcctaatcaggagtatgctgatgtctttcacttgtatttacaatacattagaaagttacatgatgagcttactggccacatgttggacatgtattccgtaatgctaactaatgataacacctcacggtactgggaacctgagttttatgaggctatgtacacaccacatacagtcttgcaggctgtaggtgcttgtgtattgtgcaattcacagacttcacttcgttgcggtgcctgtattaggagaccattcctatgttgcaagtgctgctatgaccatgtcatttcaacatcacacaaattagtgttgtctgttaatccctatgtttgcaatgccccaggttgtgatgtcactgatgtgacacaactgtatctaggaggtatgagctattattgcaagtcacataagcctcccattagttttccattatgtgctaatggtcaggtttttggtttatacaaaaacacatgtgtaggcagtgacaatgtcactgacttcaatgcgatagcaacatgtgattggactaatgctggcgattacatacttgccaacacttgtactgagagactcaagcttttcgcagcagaaacgctcaaagccactgaggaaacatttaagctgtcatatggtattgccactgtacgcgaagtactctctgacagagaattgcatctttcatgggaggttggaaaacctagaccaccattgaacagaaactatgtctttactggttaccgtgtaactaaaaatagtaaagtacagattggagagtacacctttgaaaaaggtgactatggtgatgctgttgtgtacagaggtactacgacatacaagttgaatgttggtgattactttgtgttgacatctcacactgtaatgccacttagtgcacctactctagtgccacaagagcactatgtgagaattactggcttgtacccaacactcaacatctcagatgagttttctagcaatgttgcaaattatcaaaaggtcggcatgcaaaagtactctacactccaaggaccacctggtactggtaagagtcattttgccatcggacttgctctctattacccatctgctcgcatagtgtatacggcatgctctcatgcagctgttgatgccctatgtgaaaaggcatcaaaatatttgcccatagataaatgtagtagaatcatacctgcgcgtgcgcgcgtagagtgttttgataaattcaaagtgaattcaacactagaacagtatgttttctgcactgtaaatgcattgccagaaacaactgctgacattgtagtctttgatgaaatctctatggctactaattatgacttgagtgttgtcaatgctagacttcgtgcaaaacactacgtctatattggcgatcctgctcaattaccagccccccgcacattgctgactaaaggcacactagaaccagaatattttaattcagtgtgcagacttatgaaaacaataggtccagacatgttccttggaacttgtcgccgttgtcctgctgaaattgttgacactgtgagtgctttagtttatgacaataagctaaaagcacacaaggagaagtcagctcaatgcttcaaaatgttctacaaaggtgttattacacatgatgtttcatctgcaatcaacagacctcaaataggcgttgtaagagaatttcttacacgcaatcctgcttggagaaaagctgtttttatctcaccttataattcacagaacgctgtagcttcaaaaatcttaggattgcctacgcagactgttgattcatcacagggttctgaatatgactatgtcatattcacacaaactactgaaacagcacactcttgtaatgtcaaccgcttcaatgtggctatcacaagggcaaaaattggcattttgtgcataatgtctgatagagatctttatgacaaactgcaatttacaagtctagaaataccacgtcgcaatgtggctacattacaagcagaaaatgtaactggactttttaaggactgtagtaagatcattactggtcttcatcctacacaggcacctacacacctcagcgttgatataaagttcaagactgaaggattatgtgttgacataccaggcataccaaaggacatgacctaccgtagactcatctctatgatgggtttcaaaatgaattaccaagtcaatggttaccctaatatgtttatcacccgcgaagaagctattcgtcacgttcgtgcgtggattggctttgatgtagagggctgtcatgcaactagagatgctgtgggtactaacctacctctccagctaggattttctacaggtgttaacttagtagctgtaccgactggttatgttgacactgaaaataacacagaattcaccagagttaatgcaaaacctccaccaggtgaccagtttaaacatcttataccactcatgtataaaggcttgccctggaatgtagtgcgtattaagatagtacaaatgctcagtgatacactgaaaggattgtcagacagagtcgtgttcgtcctttgggcgcatggctttgagcttacatcaatgaagtactttgtcaagattggacctgaaagaacgtgttgtctgtgtgacaaacgtgcaacttgcttttctacttcatcagatacttatgcctgctggaatcattctgtgggttttgactatgtctataacccatttatgattgatgttcagcagtggggctttacgggtaaccttcagagtaaccatgaccaacattgccaggtacatggaaatgcacatgtggctagttgtgatgctatcatgactagatgtttagcagtccatgagtgctttgttaagcgcgttgattggtctgttgaataccctattataggagatgaactgagggttaattctgcttgcagaaaagtacaacacatggttgtgaagtctgcattgcttgctgataagtttccagttcttcatgacattggaaatccaaaggctatcaagtgtgtgcctcaggctgaagtagaatggaagttctacgatgctcagccatgtagtgacaaagcttacaaaatagaggaactcttctattcttatgctacacatcacgataaattcactgatggtgtttgtttgttttggaattgtaacgttgatcgttacccagccaatgcaattgtgtgtaggtttgacacaagagtcttgtcaaacttgaacttaccaggctgtgatggtggtagtttgtatgtgaataagcatgcattccacactccagctttcgataaaagtgcatttactaatttaaagcaattgcctttcttttactattctgatagtccttgtgagtctcatggcaaacaagtagtgtcggatattgattatgttccactcaaatctgctacgtgtattacacgatgcaatttaggtggtgctgtttgcagacaccatgcaaatgagtaccgacagtacttggatgcatataatatgatgatttctgctggatttagcctatggatttacaaacaatttgatacttataacctgtggaatacatttaccaggttacagagtttagaaaatgtggcttataatgttgttaataaaggacactttgatggacacgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtatctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttcagtcatggacaacttggcggtcttcatttaatgataggcttagccaagcgctcacaagattcaccacttaaattagaggattttatccctatggacagcacagtgaaaaattacttcataacagatgcgcaaacaggttcatcaaaatgtgtgtgttctgtgattgatcttttacttgatgactttgtcgagataataaagtcacaagatttgtcagtgatttcaaaagtggtcaaggttacaattgactatgctgaaatttcattcatgctttggtgtaaggatggacatgttgaaaccttctacccaaaactacaagcaagtcaagcgtggcaaccaggtgttgcgatgcctaacttgtacaagatgcaaagaatgcttcttgaaaagtgtgaccttcagaattatggtgaaaatgctgttataccaaaaggaataatgatgaatgtcgcaaagtatactcaactgtgtcaatacttaaatacacttactttagctgtaccctacaacatgagagttattcactttggtgctggctctgataaaggagttgcaccaggtacagctgtactcagacaatggttgccaactggcacactacttgtcgattcagatcttaatgacttcgtctccgacgcagattctactttaattggagactgtgcaacagtacatacggctaataaatgggaccttattattagcgatatgtatgaccctaagaccaaacatgtgacaaaagagaatgactctaaagaagggtttttcacttatctgtgtggatttataaagcaaaaactagccctgggtggttctatagctgtaaagataacagagcattcttggaatgctgacctttacaagcttatgggccatttctcatggtggacagcttttgttacaaatgtaaatgcatcatcatcggaagcatttttaattggggctaactatcttggcaagccgaaggaacaaattgatggctataccatgcatgctaactacattttctggaggaacacaaatcctatccagttgtcttcctattcactctttgacatgagcaaatttcctcttaaattaagaggaactgctgtaatgtctcttaaggagaatcaaatcaatgatatgatttattctcttctggaaaaaggtaggcttatcattagagaaaacaacagagttgtggtttcaagtgatattcttgttaataactaaacgaacatgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttgacaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacccatttttaagttgcctcttggtattaacattacaaattttagagccattcttacagcctttttacctgctcaagacacttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaagagatgttgtgagattccctaatattacaaacttgtgtccttttggagaggtttttaatgctactaaattcccttctgtctatgcatgggagaggaaaagaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaagattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgcggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaagaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataaacgaacttatggatttgtttatgagattttttactcttggatcaattactgcacagccagtaaaaattgacaatgcttctcctgcaagtactgttcatgctacagcaacgataccgctacaagcctcactccctttcggatggcttgttattggcgttgcatttcttgctgtttttcagagcgctaccaaaataattgcgctcaataaaagatggcagctagccctttataagggcttccagttcatttgcaatttactgctgctatttgttaccatctattcacatcttttgcttgtcgctgcaggtatggaggcgcaatttttgtacctctatgccttgatatattttctacaatgcatcaacgcatgtagaattattatgagatgttggctttgttggaagtgcaaatccaagaacccattactttatgatgccaactactttgtttgctggcacacacataactatgactactgtataccatataacagtgtcacagatacaattgtcgttactgcaggtgacggcatttcaacaccaaaactcaaagaagactaccaaattggtggttattctgaggattggcactcaggtgttaaagactatgtcgttgtacatggctatttcaccgaagtttactaccagcttgagtctacacaaattactacagacactggtattgaaaatgctacattcttcatctttaacaagcttgttaaagacccaccgaatgtgcaaatacacacaatcgacggctcttcaggagttgcaaatccagcaatggatccaatttatgatgagccgacgacgactactagcgtgcctttgtaagcacaagaaagtgagtacgaacttatgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaaacgaactaactattattattattctgtttggaactttaacattgcttatcatggcagacaacggtactattaccgttgaggagcttaaacaactcctggaacaatggaacctagtaataggtttcctattcctagcctggattatgttactacaatttgcctattctaatcggaacaggtttttgtacataataaagcttgttttcctctggctcttgtggccagtaacacttgcttgctttgtgcttgctgctgtctacagaattaattgggtgactggcgggattgcgattgcaatggcttgtattgtaggcttgatgtggcttagctacttcgttgcttccttcaggctgtttgctcgtacccgctcaatgtggtcattcaacccagaaacaaacattcttctcaatgtgcctctccgggggacaattgtgaccagaccgctcatggaaagtgaacttgtcattggtgctgtgatcattcgtggtcacttgcgaatggccggacactccctagggcgctgtgacattaaggacctgccaaaagagatcactgtggctacatcacgaacgctttcttattacaaattaggagcgtcgcagcgtgtaggcactgattcaggttttgctgcatacaaccgctaccgtattggaaactataaattaaatacagaccacgccggtagcaacgacaatattgctttgctagtacagtaagtgacaacagatgtttcatcttgttgacttccaggttacaatagcagagatattgattatcattatgaggactttcaggattgctatttggaatcttgacgttataataagttcaatagtgagacaattatttaagcctctaactaagaagaattattcggagttagatgatgaagaacctatggagttagattatccataaaacgaacatgaaaattattctcttcctgacattgattgtatttacatcttgcgagctatatcactatcaggagtgtgttagaggtacgactgtactactaaaagaaccttgcccatcaggaacatacgagggcaattcaccatttcaccctcttgctgacaataaatttgcactaacttgcactagcacacactttgcttttgcttgtgctgacggtactcgacatacctatcagctgcgtgcaagatcagtttcaccaaaacttttcatcagacaagaggaggttcaacaagagctctactcgccactttttctcattgttgctgctctagtatttttaatactttgcttcaccattaagagaaagacagaatgaatgagctcactttaattgacttctatttgtgctttttagcctttctgctattccttgttttaataatgcttattatattttggttttcactcgaaatccaggatctagaagaaccttgtaccaaagtctaaacgaacatgaaacttctcattgttttgacttgtatttctctatgcagttgcatacgcactgtagtacagcgctgtgcatctaataaacctcatgtgcttgaagatccttgtcctactggttaccaacctgaatggaatataaggtacaacactaggggtaatacttatagcactgcttggctttgtgctctaggaaaggttttaccttttcatagatggcacactatggttcaaacatgcacacctaatgttactatcaactgtcaagatccagctggtggtgcgcttatagctaggtgttggtaccttcatgaaggtcaccaaactgctgcatttagagacgtatttgttgttttaaataaacgaacaaattaaaatgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaaaactgatgaagctcagcctttgccgcagagacaaaagaagcagcccactgtgactcttcttcctgcggctgacatggatgatttctccagacaacttcaaaattccatgagtggagcttctgctgattcaactcaggcataaacactcatgatgaccacacaaggcagatgggctatgtaaacgttttcgcaattccgtttacgatacatagtctactcttgtgcagaatgaattctcgtaactaaacagcacaagtaggtttagttaactttaatctcacatagcaatctttaatcaatgtgtaacattagggaggacttgaaagagccaccacattttcatcgaggccacgcggagtacgatcgagggtacagtgaataatgctagggagagctgcctatatggaagagccctaatgtgtaaaattaattttagtagtgctatccccatgtgattttaatagcttcttaggagaatgacaaaa

SEQ ID NO: 2 - DNA, 1988 bp, Sars coronaviruses (SARS-CoV)
acaggatccaagaacatgtttattttcttattatttcttactctcactagtggtagtgaccttgaccgatgcaccacttttgatgatgttcaagctcctaattacactcaacatacttcatctatgaggggggtttactatcctgatgaaatttttagatcagacactctttatttaactcaggatttatttctcccattttattctaatgttacagggtttcatactattaatcatacgtttgacaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcacgtagctttgaattgtgtgacaaccctttctttgctgtttctaaacccatgggtacacagacacatactatgatattcgataatgcatttaattgcactttcgagtacatatctgatgccctttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacccatttttaagttgcctcttggtattaacattacaaattttagagccattcttacagcctttttacctgctcaagatacttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaagagatgttgtgagattccctaatattacaaacttgtgtccttttggagaggtttttaatgctactaaattcccttctgtctatgcgtgggtgaggaaaagaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggatagaattcagatct

SEQ ID NO: 3 - DNA, 1957 bp, SARS coronaviruses (SARS-CoV)
attggatccaccatgggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattatcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgccgaacaggatcgcaacacacgtgaagtgttcgctcaagtcaaacaaatgtacaaaaccccaactttggaagattttggtggttttaatttttcacaaatattacctgaccctctaaagctaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgtcactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcctctgctaatcttgctgctactaagatgtctgagtgtgttcttggacaatcaaaaagagttgacttttgcggaaagggctaccaccttatgtccttcccacaagcagccccgcatggtgttgtcttcctacatgtcatgtatgtgccatcccaggagagaaacttcaccacagcgccagcaatttgtcatgaaggcaaagcatacttccctcgtgaaggtgtttttgtgtttaatggcacttcttggtttactacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaagaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataaacgaacttatggatttgtttatgagattttttactcttggatcaattactgcacagccagaattcggatccat

SEQ ID NO: 4 - DNA, 347 bp, SARS coronaviruses (SARS-CoV)
caaggatccgttatgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaaacgaactaactattattattattctgtttggaactttaacattgcttatcatggcagacaacggtactattaccgttgaggagcttaaagaattcagatcttgt

SEQ ID NO: 5 - DNA, 777 bp, SARS coronaviruses (SARS-CoV)
acaggatccatcatggcagacaacggtactattaccgttgaggagcttaaacaactcctggaacaatggaacctagtaataggtttcctattcctagcctggattatgttactacaatttgcctattctaatcggaacaggtttttgtacataataaagcttgttttcctctggctcttgtggccagtaacacttgcttgctttgtgcttgctgctgtctacagaattaattgggtgactggcgggattgcgattgcaatggcttgtattgtaggcttgatgtggcttagctacttcgttgcttccttcaggctgtttgctcgtacccgctcaatgtggtcattcaacccagaaacaaacattcttctcaatgtgcctctccgggggacaattgtgaccagaccgctcatggaaagtgaacttgtcattggtgctgtgatcattcgtggtcacttgcgaatggccggacactccctagggcgctgtgacattaaggacctgccaaaagagatcactgtggctacatcacgaacgctttcttattacaaattaggagcgtcgcagcgtgtaggcactgattcaggttttgctgcatacaaccgctaccgtattggaaactataaattaaatacagaccacgccggtagcaacgacaatattgctttgctagtacagtaagtgacaacagatgtttcatcttgttgacttccaggttacaatagcagagatattgattatcattatgaggactttcaggattgcgaattcagatctgtt

SEQ ID NO: 6 - DNA, 1312 bp, SARS coronaviruses (SARS-CoV)
attggatccgtcatggacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgccgccaccgtgctacaacttcctcaaggaacaacattgccaaaaggcttctacgcagagggaagcagaggcggcagccaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaagaacgtactgccacaaaacagtacaacgtcactcaagcatttgggagacgtggtccagaacagacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaaaactgatgaagctcagcctttgccgcagagacaaaagaagcagcccactgtgactcttcttcctgcggctgacatggatgatttctccagacaacttcaaaattccatgagtggagcttctgctgattcaactcaggcataaacactcatgatgaccacacaaggcagatgggctatgtaaacgttttcgcaattccgtttacgatacatagtctactcttgtgcagaatgaattcagatctgtt

SEQ ID NO: 7 - DNA, 408 bp, SARS coronaviruses (SARS-CoV)
acaccatggaattcgacatggctatttcaccgaagtttactaccagcttgagtctacacaaattactacagacactggtattgaaaatgctacattcttcatctttaacaagcttgttaaagacccaccgaatgtgcaaatacacacaatcgacggctcttcaggagttgcaaatccagcaatggatccaatttatgatgagccgacgacgactactagcgtgcctttgtaagcacaagaaagtgagtacgaacttatgtactcattcgtttcggaagaaacaggtacgttaatagttaatagcgtacttctttttcttgctttcgtggtattcttgctagtcacactagccatccttactgcgcttcgattgtgtgcgtactgctgcaatattggatccggtacctg

SEQ ID NO: 8 - protein, 7073 aa, SARS translated from nucleotides 265-21485 of SEQ ID NPO:1 coronaviruses (SARS-CoV)
MESLVLGVNEKTHVQLSLPVLQVRDVLVRGFGDSVEEALSEAREHLKNGTCGLVELEKGVLPQLEQPYVFIKRSDALSTNHCHKVVELVAEMDGIQYGRSGITLGVLVPHVGETPIAYRNVLLRKNGNKGAGGHSYGIDLKSYDLGDELGTDPIEDYEQNWNTKHGSGALRELTRELNGGAVTRYVDNNFCGPDGYPLDCIKDFLARAGKSMCTLSEQLDYIESKRGVYCCRDHEHEIAWFTERSDKSYEHQTPFEIKSAKKFDTFKGECPKFVFPLNSKVKVIQPRVEKKKTEGFMGRIRSVYPVASPQECNNMHLSTLMKCNHCDEVSWQTCDFLKATCEHCGTENLVIEGPTTCGYLPTNAVVKMPCPACQDPEIGPEHSVADYHNHSNIETRLRKGGRTRCFGGCVFAYVGCYNKRAYWVPRASADIGSGHTGITGDNVETLNEDLLEILSRERVNINIVGDFHLNEEVAIILASFSASTSAFIDTIKSLDYKSFKTIVESCGNYKVTKGKPVKGAWNIGQQRSVLTPLCGFPSQAAGVIRSIFARTLDAANHSIPDLQRAAVTILDGISEQSLRLVDAMVYTSDLLTNSVIIMAYVTGGLVQQTSQWLSNLLGTTVEKLRPIFEWIEAKLSAGVEFLKDAWEILKFLITGVFDIVKGQIQVASDNIKDCVKCFIDVVNKALEMCIDQVTIAGAKLRSLNLGEVFIAQSKGLYRQCIRGKEQLQLLMPLKAPKEVTFLEGDSHDTVLTSEEVVLKNGELEALETPVDSFTNGAIVGTPVCVNGLMLLEIKDKEQYCALSPGLLATNNVFRLKGGAPIKGVTFGEDTVWEVQGYKNVRITFELDERVDKVLNEKCSVYTVESGTEVTEFACVVAEAVVKTLQPVSDLLTNMGIDLDEWSVATFYLFDDAGEENFSSRMYCSFYPPDEEEEDDAECEEEEIDETCEHEYGTEDDYQGLPLEFGASAETVRVEEEEEEDWLDDTTEQSEIEPEPEPTPEEPVNQFTGYLKLTDNVAIKCADIVKEAQSANPMVIVNAANIHLKHGGGVAGALNKATNGAMQKESDDYIKLNGPLTVGGSCLLSGHNLAKKCLHVVGPNLNAGEDIQLLKAAYENFNSQDTLLAPLLSAGIFGAKPLQSLQVCVQTVRTQVYIAVNDKALYEQVVMDYLDNLKPRVEAPKQEEPPNTEDSKTEEKSVVQKPVDVKPKIKACIDEVTTTLEETKFLTNKLLLFADINGKLYHDSQNMLRGEDMSFLEKDAPYMVGDVITSGDITCVVIPSKKAGGTTEMLSRALKKVPVDEYITTYPGQGCAGYTLEEARTALKKCKSAFYVLPSEAPNAKEEILGTVSWNLREMLAHAEETRKLMPICMDVRAIMATIQRKYKGIKIQEGIVDYGVRFFFYTSKEPVASIITKLNSLNEPLVTMPIGYVTHGFNLEEAARCMRSLKAPAVVSVSSPDAVTTYNGYLTSSSKTSEEHFVETVSLAGSYRDWSYSGQRTELGVEFLKRGDKIVYHTLESPVEFHLDGEVLSLDKLKSLLSLREVKTIKVFTTVDNTNLHTQLVDMSMTYGQQFGPTYLDGADVTKIKPHVNHEGKTFFVLPSDDTLRSEAFEYYHTLDESFLGRYMSALNHTKKWKFPQVGGLTSIKWADNNCYLSSVLLALQQIEVKFNAPALQEAYYRARAGDAANFCALILAYSNKTVGELGDVRETMTHLLQHANLESAKRVLNVVCKHCGQKTTTLTGVEAVMYMGTLSYDNLKTGVSIPCVCGRDATQYLVQQESSFVMMSAPPAEYKLQQGTFLCANEYTGNYQCGHYTHITAKETLYRIDGAHLTKMSEYKGPVTDVFYKETSYTTTIKPVSYKLDGVTYTEIEPKLDGYYKKDNAYYTEQPIDLVPTQPLPNASFDNFKLTCSNTKFADDLNQMTGFTKPASRELSVTFFPDLNGDVVAIDYRHYSASFKKGAKLLHKPIVWHINQATTKTTFKPNTWCLRCLWSTKPVDTSNSFEVLAVEDTQGMDNLACESQQPTSEEVVENPTIQKEVIECDVKTTEVVGNVILKPSDEGVKVTQELGHEDLMAAYVENTSITIKKPNELSLALGLKTIATHGIAAINSVPWSKIFAYVKPFLGQAAITTSNCAKRLAQRVFNNYMPYVFTLLFQLCTFTKSTNSRIRASLPTTIAKNSVKSVAKLCLDAGINYVKSPKFSKLFTIAMWLLLLSICLGSLIYVTAAFGVLLSNFGAPSYCNGVRELYLNSSNVTTMDFCEGSFPCSICLSGLDSLDSYPALETIQVTISSYKLDLTILGLAAEWVLAYMLFTKFFYLLGLSAIMQVFFGYFASHFISNSWLMWFIISIVQMAPVSAMVRMYIFFASFYYIWKSYVHIMDGCTSSTCMMCYKRNRATRVECTTIVNGMKRSFYVYANGGRGFCKTHNWNCLNCDTFCTGSTFISDEVARDLSLQFKRPINPTDQSSYIVDSVAVKNGALHLYFDKAGQKTYERHPLSHFVNLDNLRANNTKGSLPINVIVFDGKSKCDESASKSASVYYSQLMCQPILLLDQALVSDVGDSTEVSVKMFDAYVDTFSATFSVPMEKLKALVATAHSELAKGVALDGVLSTFVSAARQGVVDTDVDTKDVIECLKLSHHSDLEVTGDSCNNFMLTYNKVENMTPRDLGACIDCNARHINAQVAKSHNVSLIWNVKDYMSLSEQLRKQIRSAAKKNNIPFRLTCATTRQVVNVITTKISLKGGKIVSTWFKLMLKATLLCVLAALVCYIVMPVHTLSIHDGYTNEIIGYKAIQDGVTRDIISTDDCFANKHAGFDAWFSQRGGSYKNDKSCPVVAAIITREIGFIVPGLPGTVLRAINGDFLHFLPRVFSAVGNICYTPSKLIEYSDFATSACVLAAECTIFKDAMGKPVPYCYDTNLLEGSISYSELRPDTRYVLMDGSIIQFPNIYLEGSVRVVTTFDAEYCRHGTCERSEAGICLSTSGRWVLNNEHYRALSGVFCGVDAMNLIANIFTPLVQPVGALDVSASVVAGGIIAILVTCAAYYFMKFRRAFGEYNHVVAANALLFLMSFTILCLAPAYSFLPGVYSVFYLYLTFYFTNDVSFLAHLQWFAMFSPIVPFWITAIYVFCISLKHCHWFFNNYLRKRVMFNGVTFSTFEEAALCTFLLNKEMYLKLRSETLLPLTQYNRYLALYNKYKYFSGALDTTSYREAACCHLAKALNDFSNSGADVLYQPPQTSITSAVLQSGFRKMAFPSGKVEGCMVQVTCGTTTLNGLWLDDTVYCPRHVICTAEDMLNPNYEDLLIRKSNHSFLVQAGNVQLRVIGHSMQNCLLRLKVDTSNPKTPKYKFVRIQPGQTFSVLACYNGSPSGVYQCAMRPNHTIKGSFLNGSCGSVGFNIDYDCVSFCYMHHMELPTGVHAGTDLEGKFYGPFVDRQTAQAAGTDTTITLNVLAWLYAAVINGDRWFLNRFTTTLNDFNLVAMKYNYEPLTQDHVDILGPLSAQTGIAVLDMCAALKELLQNGMNGRTILGSTILEDEFTPFDVVRQCSGVTFQGKFKKIVKGTHHWMLLTFLTSLLILVQSTQWSLFFFVYENAFLPFTLGIMAIAACAMLLVKHKHAFLCLFLLPSLATVAYFNMVYMPASWVMRIMTWLELADTSLSGYRLKDCVMYASALVLLILMTARTVYDDAARRVWTLMNVITLVYKVYYGNALDQAISMWALVISVTSNYSGVVTTIMFLARAIVFVCVEYYPLLFITGNTLQCIMLVYCFLGYCCCCYFGLFCLLNRYFRLTLGVYDYLVSTQEFRYMNSQGLLPPKSSIDAFKLNIKLLGIGGKPCIKVATVQSKMSDVKCTSVVLLSVLQQLRVESSSKLWAQCVQLHNDILLAKDTTEAFEKMVSLLSVLLSMQGAVDINRLCEEMLDNRATLQAIASEFSSLPSYAAYATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVVPDYGTYKNTCDGNTFTYASALWEIQQVVDADSKIVQLSEINMDNSPNLAWPLIVTALRANSAVKLQNNELSPVALRQMSCAAGTTQTACTDDNALAYYNNSKGGRFVLALLSDHQDLKWARFPKSDGTGTIYTELEPPCRFVTDTPKGPKVKYLYFIKGLNNLNRGMVLGSLAATVRLQAGNATEVPANSTVLSFCAFAVDPAKAYKDYLASGGQPITNCVKMLCTHTGTGQAITVTPEANMDQESFGGASCCLYCRCHIDHPNPKGFCDLKGKYVQIPTTCANDPVGFTLRNTVCTVCGMWKGYGCSCDQLREPLMQSADASTFLNRVCGVSAARLTPCGTGTSTDVVYRAFDIYNEKVAGFAKFLKTNCCRFQEKDEEGNLLDSYFVVKRHTMSNYQHEETIYNLVKDCPAVAVHDFFKFRVDGDMVPHISRQRLTKYTMADLVYALRHFDEGNCDTLKEILVTYNCCDDDYFNKKDWYDFVENPDILRVYANLGERVRQSLLKTVQFCDAMRDAGIVGVLTLDNQDLNGNWYDFGDFVQVAPGCGVPIVDSYYSLLMPILTLTRALAAESHMDADLAKPLIKWDLLKYDFTEERLCLFDRYFKYWDQTYHPNCINCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVETPHLMGWDYPKCDRAMPNMLRIMASLVLARKHNTCCNLSHRFYRLANECAQVLSEMVMCGGSLYVKPGGTSSGDATTAYANSVFNICQAVTANVNALLSTDGNKIADKYVRNLQHRLYECLYRNRDVDHEFVDEFYAYLRKHFSMMILSDDAVVCYNSNYAAQGLVASIKNFKAVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQAVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKASKYLPIDKCSRIIPARARVECFDKFKVNSTLEQYVFCTVNALPETTADIVVFDEISMATNYDLSVVNARLRAKHYVYIGDPAQLPAPRTLLTKGTLEPEYFNSVCRLMKTIGPDMFLGTCRRCPAEIVDTVSALVYDNKLKAHKEKSAQCFKMFYKGVITHDVSSAINRPQIGVVREFLTRNPAWRKAVFISPYNSQNAVASKILGLPTQTVDSSQGSEYDYVIFTQTTETAHSCNVNRFNVAITRAKIGILCIMSDRDLYDKLQFTSLEIPRRNVATLQAENVTGLFKDCSKIITGLHPTQAPTHLSVDIKFKTEGLCVDIPGIPKDMTYRRLISMMGFKMNYQVNGYPNMFITREEAIRHVRAWIGFDVEGCHATRDAVGTNLPLQLGFSTGVNLVAVPTGYVDTENNTEFTRVNAKPPPGDQFKHLIPLMYKGLPWNVVRIKIVQMLSDTLKGLSDRVVFVLWAHGFELTSMKYFVKIGPERTCCLCDKRATCFSTSSDTYACWNHSVGFDYVYNPFMIDVQQWGFTGNLQSNHDQHCQVHGNAHVASCDAIMTRCLAVHECFVKRVDWSVEYPIIGDELRVNSACRKVQHMVVKSALLADKFPVLHDIGNPKAIKCVPQAEVEWKFYDAQPCSDKAYKIEELFYSYATHHDKFTDGVCLFWNCNVDRYPANAIVCRFDTRVLSNLNLPGCDGGSLYVNKHAFHTPAFDKSAFTNLKQLPFFYYSDSPCESHGKQVVSDIDYVPLKSATCITRCNLGGAVCRHHANEYRQYLDAYNMMISAGFSLWIYKQFDTYNLWNTFTRLQSLENVAYNVVNKGHFDGHAGEAPVSIINNAVYTKVDGIDVEIFENKTTLPVNVAFELWAKRNIKPVPEIKILNNLGVDIAANTVIWDYKREAPAHVSTIGVCTMTDIAKKPTESACSSLTVLFDGRVEGQVDLFRNARNGVLITEGSVKGLTPSKGPAQASVNGVTLIGESVKTQFNYFKKVDGIIQQLPETYFTQSRDLEDFKPRSQMETDFLELAMDEFIQRYKLEGYAFEHIVYGDFSHGQLGGLHLMIGLAKRSQDSPLKLEDFIPMDSTVKNYFITDAQTGSSKCVCSVIDLLLDDFVEIIKSQDLSVISKVVKVTIDYAEISFMLWCKDGHVETFYPKLQASQAWQPGVAMPNLYKMQRMLLEKCDLQNYGENAVIPKGIMMNVAKYTQLCQYLNTLTLAVPYNMRVIHFGAGSDKGVAPGTAVLRQWLPTGTLLVDSDLNDFVSDADSTLIGDCATVHTANKWDLIISDMYDPKTKHVTKENDSKEGFFTYLCGFIKQKLALGGSIAVKITEHSWNADLYKLMGHFSWWTAFVTNVNASSSEAFLIGANYLGKPKEQIDGYTMHANYIFWRNTNPIQLSSYSLFDMSKFPLKLRGTAVMSLKENQINDMIYSLLEKGRLIIRENNRVVVSSDILVNN

SEQ ID NO: 9 - protein, 653 aa, SARS Coronaviruses (SARS-CoV)
MFIFLLFLTLTSGSDLDRCTTFDDVQAPNYTQHTSSMRGVYYPDEIFRSDTLYLTQDLFLPFYSNVTGFHTINHTFDNPVIPFKDGIYFAATEKSNVVRGWVFGSTMNNKSQSVIIINNSTNVVIRARSFELCDNPFFAVSKPMGTQTHTMIFDNAFNCTFEYISDALSLDVSEKSGNFKHLREFVFKNKDGFLYVYKGYQPIDVVRDLPSGFNTLKPIFKLPLGINITNFRAILTAFLPAQDTWGTSAAAYFVGYLKPTTFMLKYDENGTITDAVDCSQNPLAELKCSVKSFEIDKGIYQTSNFRVVPSRDVVRFPNITNLCPFGEVFNATKFPSVYAWVRKRISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIG

SEQ ID NO: 10 - protein, 623 aa, SARS Coronaviruses (SARS-CoV)
MGCLIGAEHVDTSYECDIPIGAGICASYHTVSLLRSTSQKSIVAYTMSLGADSSIAYSNNTIAIPTNFSIIITTEVMPVSMAKTSVDCNMYICGDSTECANLLLQYGSFCTQLNRALSGIAAEQDRNTREVFAQVKQMYKTPTLEDFGGFNFSQILPDPLKLTKRSFIEDLLFNKVTLADAGFMKQYGECLGDINARDLICAQKFNGLTVLPPLLTDDMIAAYTAALVSGTVTAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVMYVPSQERNFTTAPAICHEGKAYFPREGVFVFNGTSWFTTQRNFFSPQIITTDNTFVSGNCDVVIGIINNTVYDPLQPELDSFKEELDKYFKNHTSPDVDLGDISGINASVVNIQEEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 11 - protein, 76 aa, SARS Coronaviruses (SARS-CoV)
MYSFVSEETGTLIVNSVLLFLAFVVFLLVTLAILTALRLCAYCCNIVNVSLVKPTVYVYSRVKNLNSSEGVPDLLV

SEQ ID NO: 12 - protein, 221 aa, SARS Coronaviruses (SARS-CoV)
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHSLGRCDIKDLPKEITVATSRTLSYYKLGASQRVGTDSGFAAYNRYRIGNYKLNTDHAGSNDNIALLVQ

SEQ ID NO: 13 - protein, 398 aa, SARS Coronaviruses (SARS-CoV)
MDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQERTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 14 - protein, 91 aa, SARS Coronaviruses (SARS-CoV)
MAISPKFTTSLSLHKLLQTLVLKMLHSSSLTSLLKTHRMCKYTQSTALQELQIQQWIQFMMSRRRLLACLCKHKKVSTNLCTHSFRKKQVR

SEQ ID NO: 15 - DNA, 29 bp, Aritifical Sequence
  Synthetic primer
cctactggttaccaacctgaatggaatat

SEQ ID NO: 16 - DNA, 32 bp, Artificial Sequence
  Synthetic primer
acaggatccaagaacatgtttattttcttatt

SEQ ID NO: 17 - DNA, 35 bp, Artificial Sequence
  Synthentic primer
agatctgaattctatccaataggaatgtcgcactc

SEQ ID NO: 18 - DNA, 37 bp, Artificial Sequence
  Synthetic primer
attggatccaccatgggctgtcttataggagctgagc

SEQ ID NO: 19 - DNA, 35 bp, Artificial Sequence
  Synthetic primer
atggatccgaattctggctgtgcagtaattgatct

SEQ ID NO: 20 - DNA, 30 bp, Artificial Sequence
  Synthetic primer
caaggatccgttatgtactcattcgtttcg

SEQ ID NO: 21 - DNA, 35 bp, Artificial Sequence
  Synthetic primer
acaagatctgaattctttaagctcctcaacggtaa

SEQ ID NO: 22 - DNA, 29 bp, Artificial Sequence
  Synthetic primer
acaggatccatcatggcagacaacggtac

SEQ ID NO: 23 - DNA, 36 bp, Artificial Sequence
  Synthetic primer
aacagatctgaattcgcaatcctgaaagtcctcata

SEQ ID NO: 24 - DNA, 38 bp, Artificial Sequence
  Synthetic primer
attggatccgtcatggacaataaccagaatggaggacg

SEQ ID NO: 25 - DNA, 29 bp, Artificial Sequence
  Synthetic primer
aacagatctgaattcattctgcacaagag

SEQ ID NO: 26 - DNA, 35 bp, Artificial Sequence
  Synthetic primer
acaccatggaattcgacatggctatttcaccgaag

SEQ ID NO: 27 - DNA, 34 bp, Artificial Sequence
  Synthetic pimer
caggtaccggatccaatattgcagcagtacgcac

SEQ ID NO: 28 - protein, 1255 aa, SARS translated from nucleotides 21492-25259 of SEQ ID NO:1 coronaviruses (SARS-CoV)
MFIFLLFLTLTSGSDLDRCTTFDDVQAPNYTQHTSSMRGVYYPDEIFRSDTLYLTQDLFLPFYSNVTGFHTINHTFDNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFLPAQDTWGTSAAAYFVGYLKPTTFMLKYDENGTITDAVDCSQNPLAELKCSVKSFEIDKGIYQTSNFRVVPSRDVVRFPNITNLCPFGEVFNATKFPSVYAWERKRISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKDFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQEEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 29 - protein, 274 aa, SARS translated from nucleotides 25268-26092 of SEQ ID NO:1 coronaviruses (SARS-CoV)
MDLFMRFFTLGSITAQPVKIDNASPASTVHATATIPLQASLPFGWLVIGVAFLAVFQSATKIIALNKRWQLALYKGFQFICNLLLLFVTIYSHLLLVAAGMEAQFLYLYALIYFLQCINACRIIMRCWLCWKCKSKNPLLYDANYFVCWHTHNYDYCIPYNSVTDTIVVTAGDGISTPKLKEDYQIGGYSEDWHSGVKDYVVVHGYFTEVYYQLESTQITTDTGIENATFFIFNKLVKDPPNVQIHTIDGSSGVANPAMDPIYDEPTTTTSVPL

SEQ ID NO: 30 - protein, 154 aa, SARS translated from nucleotides 25689-26153 of SEQ ID NO:1 coronaviruses (SARS-CoV))
MMPTTLFAGTHITMTTVYHITVSQIQLSLLQVTAFQHQNSKKTTKLVVILRIGTQVLKTMSLYMAISPKFTTSLSLHKLLQTLVLKMLHSSSLTSLLKTHRMCKYTQSTALQELQIQQWIQFMMSRRRLLACLCKHKKVSTNLCTHSFRKKQVR

SEQ ID NO: 31 - protein, 76 aa, SARS translated from nucleotides 26117-26347 of SEQ ID NO:1 coronaviruses (SARS-CoV); identical to SEQ ID NO: 11

SEQ ID NO: 32 - protein, 218 aa, SARS translated from nucleotides 26398-27063 of SEQ ID NO:1 coronaviruses (SARS-CoV)
MADNGTITVEELKQLLEQWNLVIGFLFLAWIMLLQFAYSNRNRFLYIIKLVFLWLLWPVTLACFVLAAVYRINWVTGGIAIAMACIVGLMWLSYFVASFRLFARTRSMWSFNPETNILLNVPLRGTIVTRPLMESELVIGAVIIRGHLRMAGHSLGRCDIKDLPKEITVATSRTLSYYKLGASQRVGTDSGFAAYNRYRIGNYKLNTDHAGSNDNIAL

SEQ ID NO: 33 - protein, 63 aa, SARS translated from nucleotides 27074-27265 of SEQ ID NO:1 coronaviruses (SARS-CoV)
MFHLVDFQVTIAEILIIIMRTFRIAIWNLDVIISSIVRQLFKPLTKKNYSELDDEEPMELDYP

SEQ ID NO: 34 - protein, 44 aa, SARS translated from nucleotides 27638-27772 of SEQ ID NO:1 coronaviruses (SARS-CoV)
MNELTLIDFYLCFLAFLLFLVLIMLIIFWFSLEIQDLEEPCTKV

SEQ ID NO: 35 - protein, 122 aa, SARS translated from nucleotides 27779-28147 of SEQ ID NO:1 coronaviruses (SARS-CoV)
MKLLIVLTCISLCSCIRTVVQRCASNKPHVLEDPCPTGYQPEWNIRYNTRGNTYSTAWLCALGKVLPFHRWHTMVQTCTPNVTINCQDPAGGALIARCWYLHEGHQTAAFRDVFVVLNKRTN

SEQ ID NO: 36 - protein, 422 aa, SARS translated from nucleotides 28149-29417 of SEQ ID NO:1 coronaviruses (SARS-CoV)
MSDNGPQSNQRSAPRITFGGPTDSTDNNQNGGRNGARPKQRRPQGLPNNTASWFTALTQHGKEELRFPRGQGVPINTNSGPDDQIGYYRRATRRVRGGDGKMKELSPRWYFYYLGTGPEASLPYGANKEGIVWVATEGALNTPKDHIGTRNPNNNAATVLQLPQGTTLPKGFYAEGSRGGSQASSRSSSRSRGNSRNSTPGSSRGNSPARMASGGGETALALLLLDRLNQLESKVSGKGQQQQGQTVTKKSAAEASKKPRQKRTATKQYNVTQAFGRRGPEQTQGNFGDQDLIRQGTDYKHWPQIAQFAPSASAFFGMSRIGMEVTPSGTWLTYHGAIKLDDKDPQFKDNVILLNKHIDAYKTFPPTEPKKDKKKKTDEAQPLPQRQKKQPTVTLLPAADMDDFSRQLQNSMSGASADSTQA

SEQ ID NO: 37 - protein, 98 aa, SARS translated from nucleotides 28159-28455 of SEQ ID NO:1 coronaviruses (SARS-CoV)
MDPNQTNVVPPALHLVDPQIQLTITRMEDAMGQGQNSADPKVYPIILRLGSQLSLSMARRNLDSLEARAFQSTPIVVQMTKLATTEELPDEFVVVTAK